SEQUENCE LISTING (50 sequences)

SEQ ID NO: 1 - DNA, 20 bp, Artificial Sequence
  Synthetic DNA
gatgcctctgattggtctgg

SEQ ID NO: 2 - DNA, 21 bp, Artificial Sequence
  Synthetic DNA
cttggatgtcacaaacagctc

SEQ ID NO: 3 - DNA, 21 bp, Artificial Sequence
  Synthetic DNA
caactttgaggccaataccac

SEQ ID NO: 4 - DNA, 20 bp, Artificial Sequence
  Synthetic DNA
tccttgctccaggcaagatg

SEQ ID NO: 5 - DNA, 21 bp, Artificial Sequence
  Synthetic DNA
tcaatgatgggaaggtggatc

SEQ ID NO: 6 - DNA, 21 bp, Artificial Sequence
  Synthetic DNA
aggtcatagtcaaaggcatcc

SEQ ID NO: 7 - DNA, 20 bp, Artificial Sequence
  Synthetic DNA
tggctcttgggtttgagaag

SEQ ID NO: 8 - DNA, 21 bp, Artificial Sequence
  Synthetic DNA
catcttggaactgggaatacg

SEQ ID NO: 9 - DNA, 20 bp, Artificial Sequence
  Synthetic DNA
atcttcttctgtgccacgcc

SEQ ID NO: 10 - DNA, 20 bp, Artificial Sequence
  Synthetic DNA
gctcccattgctttggccac

SEQ ID NO: 11 - DNA, 20 bp, Artificial Sequence
  Synthetic DNA
atgcctgctgacttgccttc

SEQ ID NO: 12 - DNA, 20 bp, Artificial Sequence
  Synthetic DNA
ctgaggctcttccacaagag

SEQ ID NO: 13 - DNA, 22 bp, Artificial Sequence
  Synthetic DNA
aagaagatcctctgtgaagaac

SEQ ID NO: 14 - DNA, 21 bp, Artificial Sequence
  Synthetic DNA
gaatgctgaagatgttggcag

SEQ ID NO: 15 - DNA, 19 bp, Artificial Sequence
  Synthetic DNA
ggtgccactgtccatgagc

SEQ ID NO: 16 - DNA, 20 bp, Artificial Sequence
  Synthetic DNA
ggcggtagttgtaggcaatg

SEQ ID NO: 17 - DNA, 19 bp, Artificial Sequence
  Synthetic DNA
gacagcggcagagactatg

SEQ ID NO: 18 - DNA, 19 bp, Artificial Sequence
  Synthetic DNA
ccaccttctggcggtagag

SEQ ID NO: 19 - DNA, 20 bp, Artificial Sequence
  Synthetic DNA
tcctgaccactggcatgaac

SEQ ID NO: 20 - DNA, 20 bp, Artificial Sequence
  Synthetic DNA
tgctcagagccatgatcatc

SEQ ID NO: 21 - DNA, 20 bp, Artificial Sequence
  Synthetic DNA
gagcagcaacctcaagagag

SEQ ID NO: 22 - DNA, 21 bp, Artificial Sequence
  Synthetic DNA
cgaggatcttcttaaactgag

SEQ ID NO: 23 - DNA, 20 bp, Artificial Sequence
  Synthetic DNA
tgtgtctgcagtgctgtgtg

SEQ ID NO: 24 - DNA, 20 bp, Artificial Sequence
  Synthetic DNA
agacacaccttccaagcatc

SEQ ID NO: 25 - DNA, 20 bp, Artificial Sequence
  Synthetic DNA
ggaacaatatcctgcactcc

SEQ ID NO: 26 - DNA, 22 bp, Artificial Sequence
  Synthetic DNA
ctgtaattaatacattgcctcc

SEQ ID NO: 27 - DNA, 20 bp, Artificial Sequence
  Synthetic DNA
ggagcagctggtcaaattgc

SEQ ID NO: 28 - DNA, 20 bp, Artificial Sequence
  Synthetic DNA
tccatgccttcccttcttgg

SEQ ID NO: 29 - DNA, 19 bp, Artificial Sequence
  Synthetic DNA
gctggtggaagagggcttc

SEQ ID NO: 30 - DNA, 20 bp, Artificial Sequence
  Synthetic DNA
cctttgcagtctggcaagtg

SEQ ID NO: 31 - DNA, 20 bp, Artificial Sequence
  Synthetic DNA
gttgctggtcacattcctgg

SEQ ID NO: 32 - DNA, 21 bp, Artificial Sequence
  Synthetic DNA
taggccttcaactccttcatg

SEQ ID NO: 33 - DNA, 20 bp, Artificial Sequence
  Synthetic DNA
agttccaggacggagacctc

SEQ ID NO: 34 - DNA, 21 bp, Artificial Sequence
  Synthetic DNA
cagggtctcaaaaggcttcag

SEQ ID NO: 35 - DNA, 19 bp, Artificial Sequence
  Synthetic DNA
agtcatccctgtgcgcatc

SEQ ID NO: 36 - DNA, 19 bp, Artificial Sequence
  Synthetic DNA
ggtccaccttggtgtagtc

SEQ ID NO: 37 - DNA, 20 bp, Artificial Sequence
  Synthetic DNA
tctccaaataccagctcgac

SEQ ID NO: 38 - DNA, 20 bp, Artificial Sequence
  Synthetic DNA
caggatcttgcccttgaagc

SEQ ID NO: 39 - DNA, 21 bp, Artificial Sequence
  Synthetic DNA
gtccagctgcaacatcttctc

SEQ ID NO: 40 - DNA, 20 bp, Artificial Sequence
  Synthetic DNA
cagtcgtggtctcctcagag

SEQ ID NO: 41 - DNA, 21 bp, Artificial Sequence
  Synthetic DNA
gatgaaggaaattgcagaagc

SEQ ID NO: 42 - DNA, 22 bp, Artificial Sequence
  Synthetic DNA
caatagtgaggattgacacatc

SEQ ID NO: 43 - DNA, 19 bp, Artificial Sequence
  Synthetic DNA
caggattctcctgggtgac

SEQ ID NO: 44 - DNA, 19 bp, Artificial Sequence
  Synthetic DNA
gatgtagagaccttctctg

SEQ ID NO: 45 - DNA, 21 bp, Artificial Sequence
  Synthetic DNA
cttcagcaactggcttgcatg

SEQ ID NO: 46 - DNA, 20 bp, Artificial Sequence
  Synthetic DNA
gctgagctggatcatctgag

SEQ ID NO: 47 - DNA, 20 bp, Artificial Sequence
  Synthetic DNA
agcccaagtgtgtggaagag

SEQ ID NO: 48 - DNA, 20 bp, Artificial Sequence
  Synthetic DNA
tgctggttgctcaccatgtc

SEQ ID NO: 49 - DNA, 21 bp, Artificial Sequence
  Synthetic DNA
cctggaatacacagactcaag

SEQ ID NO: 50 - DNA, 20 bp, Artificial Sequence
  Synthetic DNA
tcttctcctcttctgtctcc

(19) United States
(12) Patent Application Publication
(10) Pub. No.: US 2009/0181379 A1
(43) Pub. Date: Jul. 16, 2009

(54) MOLECULAR MARKERS OF HEPATOCELLULAR CARCINOMA AND THEIR APPLICATIONS

(75) Inventors: Fernando Corrales Izquierdo, Pamplona (ES); Enrique Santamaria Martinez, Pamplona (ES); Javier Munoz Peralta, Pamplona (ES); Jesus Prieto Valtuena, Pamplano (ES); Matias Avila Zaragoza, Pamplano (ES)

Correspondence Address: MOORE & VAN ALLEN PLLC, P.O. BOX 13706, Research Triangle Park, NC, 27709, US

(73) Assignee: PROYECTO DE BIOMEDICINA CIMA, S.L., Cizur Mayor (ES)

(21) Appl. No.: 12/088,577

(22) PCT Filed: Sep. 28, 2006
(86) PCT No.: PCT/EP2006/066863

§ 371 Date: Aug. 28, 2008

(30) Foreign Application Priority Data
Sep. 29, 2005 (ES) P200502369

(51) Int. Cl. C12Q 1/68; C12Q 1/32; C12Q 1/34; C12Q 1/52

(52) U.S. Cl. 435/6; 435/26; 435/18; 435/16

(57) ABSTRACT

By means of a proteomic approach, the markers of hepatocellular carcinoma (HCC) in the liver of a knockout mouse (MAT1A−/−) have been identified for the MAT1A gene (deficient in the synthesis of S-adenosylmethionine). 27 proteins have been detected the expression thereof is altered in, at least, 50% of the analysed tumours. Amongst them, 13 proteins have been validated in biopsies of patients with HCC of different etiology, and 7 of them have been validated in biopsies of patients with liver cirrhosis, a stage prior to the development of HCC, which makes it possible to differentiate between prior stages of the disease and even between different etiologies (viral and alcoholic). Having a panel of markers available may contribute to more accurately defining the alterations associated with the development of HCC and thus facilitate prognosis and diagnosis of this disease.

### FIELD OF THE INVENTION

This invention relates, in general, to the analysis of the changes in the expression of genes and proteins in liver tumour tissue from patients with hepatocellular carcinoma (HCC). In particular, the invention relates to a set of human genes and proteins which are differentially expressed in cancerous liver tissue from a subject suffering from HCC as compared to the expression of the same genes and proteins in healthy liver tissue. Said genes and proteins act, therefore, as molecular markers or biomarkers of HCC.

### BACKGROUND OF THE INVENTION

Hepatocellular carcinoma (HCC), also known as malignant hepatoma, is the neoplastic disease with the fifth highest incidence and the third cause of cancer death, with over 500,000 new cases diagnosed every year. Although the main causes of HCC are well-known, amongst them, infection by the hepatitis B virus (HBV), or the hepatitis C virus (HCV), consumption of food products contaminated with aflatoxin, or abusive alcohol consumption, the prognosis of patients with HCC is bad due to the aggressiveness of the lesion and the lack of effective therapies.

HCC is difficult to detect at early stages due to the non-specificity of the symptoms it exhibits, which include loss of appetite and weight, fever, fatigue and weakness. The only tools currently available for the early detection of HCC are measurement of serum levels of alpha-fetoprotein (AFP) and liver ultrasonography. However, AFP levels exhibit a low sensitivity and specificity, for which reason its applications as a biomarker are very limited. On the other hand, ultrasonography is dependent on the operator and has limited effectiveness when trying to differentiate HCC from regenerative nodules. Additional diagnostic methods include non-radioactive imaging diagnostic methods (X-rays, angiograms, CT scans, MRIs, etc.), liver scanners using radioactive materials or liver biopsies. Treatment of HCC is often quite inefficient due to the fact that detection takes place too late. Therapeutic methods include surgery, chemotherapy and radiotherapy, by themselves or in combination.

Therefore, the improvement of knowledge about the molecular pathogenesis of HCC and the identification of biomarkers which allow for early diagnosis have great interest and represent one of the main priorities of clinical hepatology.

The recent development of techniques such as genomics and proteomics (techniques which allow for the simultaneous analysis of thousands of genes and proteins) has led to the appearance of a series of HCC biomarkers (Lok A S, Marrero J. Newer Markers for Hepatocellular Carcinoma. Gastroenterology 2004; 127:S13-S119; Chen Li, Ye-Xiong Tan, Hu Zhou et al. Proteomic Analysis of Hepatitis B Virus Associated Hepatocellular Carcinoma: Identification of Potential Tumor Markers. Proteomics 2005; 5:1125-1139; Beretta L, Chignard N. Proteomics for Hepatocellular Carcinoma Marker Discovery. Gastroenterology 2004; 127:S120-S125; Schwegler E E, Cazares L et al. SELDI-TOF MS Profiling of Serum for Detection of the Progression of Chronic Hepatitis C to Hepatocellular Carcinoma. Hepatology 2005; 41:634-42; Yokoyama Y, Kuramitsu Y et al. Proteomic Profiling of Proteins Decreased in Hepatocellular Carcinoma from Patients Infected with Hepatitis C Virus. Proteomics 2004; 4:2111-6; Thorgeirsson S et al. Genome-Scale Profiling of Gene Expression in Hepatocellular Carcinoma Classification, Survival Prediction and Identification of Therapeutic Targets. Gastroenterology 2004; 127:851-855; María W. Smith, Zhaoxia N et al. Identification of Novel Tumors Markers in Hepatitis C Virus-Associated Hepatocellular Carcinoma. Cancer Research 2003; 63:859-864; Xin Chen, Siu Tim Cheung et al. Gene Expression Patterns in Human Liver Cancers. Molecular Biology of the Cell 2002; 13:1929-1939).

Using differential proteomics, a panel of proteins with a specifically altered expression pattern which are proposed as HCC biomarkers and, occasionally, have been assigned a key role in the development of the disease, has been obtained. However, the different protein repertoires identified in the different studies have little in common; for this reason, they have only relative value as HCC biomarkers. The discrepancies between the studies conducted at different laboratories might be caused by differences in the methodology used or in the nature or type of sample analysed.

Amongst the HCC biomarkers identified in different pre-clinical studies, one can find the ones listed below (translated from Lok A S, Marrero J. Newer Markers for Hepatocellular Carcinoma. Gastroenterology 2004; 127:S113-S119).

| | Biological | | |
|---|---|---|---|
| Phase I Markers | material | Sensitivity (%) | Specificity (%) |
| Glypican-3 | Tissue | 72 | 100 |
| | FERUM | 53 | |
| 73 Golgi Protein | FERUM | 76 | 69 |
| p16 methylation | FERUM | 81 | |
| Human hepatocyte | FERUM | 100 | 64 |
| growth factor | | | |
| Cytokeratin 19 | FERUM | 47 | |
| 90K-Mac-2BP | FERUM | 46 | 61 |
| glycoprotein | | | |
| TGF β1 | FERUM | 69 | 66 |
| Lipoprotein (a) | FERUM | 44 | |
| Erythrocyte-binding | FERUM | 43 | 92 |
| polyamine | | | |
| Tissue-specific | FERUM | 73 | 71 |
| polypeptide antigen | | | |
| C-reactive protein | FERUM | 48 | 58 |
| Anti-p53 antibodies | FERUM | 41 | |
| Gene CD24 | Tissue | 66 | |
| Telomerase activity | Tissue | 100 | 50 |
| Alpha-prothymosin | Tissue | 82 | |
| DNA microsatellite | Tissue | 100 | 80 |
| analysis | | | |
| Hepatocellular carcinoma | Tissue | 89 | |
| associated gene 1 | | | |
| Hematoma specific | Tissue | 85 | 97 |
| γ-glutamyltransferase | | | |
| Secretion of | Urine | 70 | |
| pseudouridine | | | |
| Epidermal growth factor | Urine | 62 | |
| receptor (EGFR) | | | |

Despite the various studies conducted in order to attempt to understand the clinical-pathological characteristics of the disease and improve the treatment of patients with HCC, conventional clinical-pathological parameters have a very limited predictive capacity. Consequently, there is still a need to identify molecular biomarkers associated with HCC. The identification of said molecular biomarkers and the study of their functional effects could help in the prevention and/or treatment of HCC, as well as in the search for and the development of drugs that are useful in the treatment, preventive and/or curative, of HCC.

### SUMMARY OF THE INVENTION

A proteomic approach has been used for the identification of markers of hepatocarcinogenesis in the liver of a knockout mouse (MAT1A−/−) for the MAT1A gene (deficient in the synthesis of S-adenosylmethionine). 27 proteins have been detected the expression thereof is altered in, at least, 50% of the analysed tumours. Amongst them, 13 proteins have been validated in biopsies of patients with HCC of different etiology, and 7 of them have been validated in biopsies of patients with liver cirrhosis, a stage prior to the development of HCC. This study has made it possible to differentiate between prior stages of the disease and even between different etiologies (viral and alcoholic); therefore, having a panel of markers available may contribute to more accurately defining the alterations associated with the development of HCC and thus facilitate prognosis and diagnosis of this disease.

For the identification of said biomarkers, the MAT1A−/− mouse has been used as a screening system prior to the analysis of human samples. Said experimental model makes it possible to conduct longitudinal studies from preneoplastic stages and to identify proteins associated with the hepatocarcinogenesis process; therefore, said experimental model is effective in the identification of HCC markers. The protein expression pattern that has been generated in this invention provides a system for a more accurate prognosis of the disease as compared to the systems described thus far, those based on biomarkers as well as on histological and clinical criteria.

It has now been found that the expression levels of certain genes (ADH, AOP2, SMP30, NSLT, SDH, SAP, PGM, P4H, APOA1, CAIII, AdDHm, OAT and/or OCT) and proteins (ADH, AOP2, SMP30, NSLT, SDH, SAP, PGM, P4H, APOA1, CAIII, AdDHm, OAT and/or OCT) are reduced in liver tissue samples from patients with HCC as compared to the expression levels of the same genes and proteins in liver tissue samples from healthy subjects, without liver disease (controls). It has also been found that the expression levels of certain genes (ADH, AOP2, SMP30, NSLT, SDH, SAP and/or PGM) and proteins (ADH, AOP2, SMP30, NSLT, SDH, SAP and/or PGM) are reduced in liver tissue samples from patients with cirrhosis, a stage prior to the development of HCC, as compared to the expression levels of the same genes and proteins in liver tissue samples from healthy subjects (controls).

Therefore, the invention relates, in general, to the use of genes ADH, AOP2, SMP30, NSLT, SDH, SAP, PGM, P4H, APOA1, CAIII, AdDHm, OAT and/or OCT, and/or the corresponding proteins (ADH, AOP2, SMP30, NSLT, SDH, SAP, PGM, P4H, APOA1, CAIII, AdDHm, OAT and/or OCT) as HCC biomarkers. The invention also relates to the use of genes ADH, AOP2, SMP30, NSLT, SDH, SAP and/or PGM, and/or the corresponding proteins (ADH, AOP2, SMP30, NSLT, SDH, SAP and/or PGM) as biomarkers of stages prior to the development of HCC. In addition, the invention relates to methods and kits for the implementation of the present invention.

In an aspect, the invention relates to an in vitro method to diagnose HCC in a subject, or to determine the stage or severity of said disease in a subject, or to monitor the effect of the therapy administered to a subject suffering from said disease, which comprises the use of said genes (ADH, AOP2, SMP30, NSLT, SDH, SAP, PGM, P4H, APOA1, CAIII, AdDHm, OAT and/or OCT) and/or proteins (ADH, AOP2, SMP30, NSLT, SDH, SAP, PGM, P4H, APOA1, CAIII, AdDHm, OAT and/or OCT) as HCC biomarkers.

In another aspect, the invention relates to an in vitro method for identifying a stage prior to the development of HCC in a subject, which comprises the use of said genes ADH, AOP2, SMP30, NSLT, SDH, SAP and/or PGM, and/or proteins (ADH, AOP2, SMP30, NSLT, SDH, SAP and/or PGM) as biomarkers of stages prior to the development of HCC.

The claims contain additional aspects related to the invention.

### BRIEF DESCRIPTION OF THE FIGURES

FIG. 1 is a set of photographs of different mice livers which show the tumour nodules analysed by means of proteomic techniques. A) 18-month-old WT liver. B) MAT1A−/− liver wherein it can be observed 2 tumour nodules approximately 5 mm in size. C) MAT1A−/− liver wherein it can be observed a 10-mm tumour nodule.

FIG. 2 shows the heterogeneity of the tumour proteome. A) Two-dimensional gel sections which illustrate the proteomic variability of the analysed tumour nodules. B) Graph which illustrates the increase in variability between tumour nodules from different MAT1A−/− mice. The percent variability was defined as the percentage of differences observed with respect to the total number of bands analysed. At least 3 independent experiments were conducted for each group.

FIG. 3 shows the linear correlation between the molecular weight (Mw) and the isoelectric point (pI) deduced on the basis of the sequence of the identified proteins, and the relative mobility (Rf) of the corresponding bands calculated on the basis of the 2D gels.

FIG. 4 shows the overall analysis of the proteins that were differentially expressed in the tumour nodules. The 151 identified proteins were classified A) on the basis of the biological process wherein they are involved and B) on the basis of their subcellular location. The columns indicate the altered proteins (differentially expressed proteins or proteins whose expression is altered) in each of the 6 WT vs tumour comparisons. Those proteins the expression thereof decreases or increases are represented in green and in red, respectively.

FIG. 5 shows the HCC-associated proteins the expression thereof is reduced in the liver of the MAT1A−/− mouse.

FIG. 6 shows the HCC-associated proteins. A) HCC-associated proteins the expression thereof increases in the liver of the MAT1A−/− mouse. B) HCC-associated proteins which exhibit an anomalous electrophoretic behaviour.

FIG. 7 shows the potential HCC biomarkers identified in the present invention. mRNA levels in control individuals and in patients with different liver diseases have been compared by means of the Mann-Whitney U statistical test. The number of asterisks refers to the significance level achieved (* indicates p<0.05; ** indicates p<0.01; *** indicates p<0.001).

### DETAILED DESCRIPTION OF THE INVENTION

### Definitions

In order to facilitate the understanding of the invention described in this patent application, the meaning of some terms and expressions in the context of the invention are explained below:

The term “subject” refers to a member of a mammal animal species, and includes, but is not limited thereto, domestic animals, primates and humans; the subject is preferably a human being, male or female, of any age or race. Alternatively, the term “individual” is also sometimes used in this description to refer to human beings.

The term “HCC” refers to hepatocellular carcinoma or malignant hepatoma.

The term “protein” refers to a molecular chain of amino acids, linked by covalent or non-covalent bonds. The term includes all the forms of post-translational modifications, for example, glycosylation, phosphorylation or acetylation.

The term “antibody” refers to a protein with the capacity to specifically bind to an “antigen”. The term “antibody” comprises recombinant antibodies, monoclonal antibodies, or polyclonal antibodies, intact, or fragments thereof which maintain the capacity to bind to the antigen, combibodies, etc., both human or humanised and of non-human origin.

The term “primer oligonucleotide”, as used in the present invention, refers to a nucleotide sequence, which is complementary to a nucleotide sequence of a selected gene. Each primer oligonucleotide hybridises with its target nucleotide sequence and acts as an initiation point for DNA polymerisation.

### Molecular Biomarkers of HCC and Applications

Many biological functions are accompanied by the alteration of the expression of various genes by means of transcription control (e.g. through control of initiation, RNA precursors, RNA processing) and/or translation control. As an illustration, certain essential biological processes, such as the cell cycle, cell differentiation and cell death, are characterised by variations in the expression levels of gene groups.

Similarly, changes in gene expression are associated with the genesis of various pathologies (pathogenesis); for example, the absence of a sufficient expression of genes which suppress functional tumours and/or overexpression of oncogenes/protooncogenes might lead towards tumorigenesis or hyperplastic cell growth. Therefore, changes in the expression of certain genes (e.g. oncogenes or tumour suppressants) may be used to evaluate the presence and progression of various pathologies.

The monitoring of changes in gene expression may also provide certain advantages during the screening and development of drugs.

Using a proteomic approach for the identification of markers of hepatocarcinogenesis in the liver of a knockout mouse (MAT1A−/−) for the MAT1A gene, the inventors have detected 27 proteins the expression thereof is altered in, at least, 50% of the analysed tumours. Amongst them, 13 proteins have been validated in biopsies of patients with HCC of different etiology, and 7 of them have been validated in biopsies of patients with liver cirrhosis, a stage prior to the development of HCC, which makes it possible to distinguish between different prior stages of the disease and even between different etiologies (viral and alcoholic); therefore, this may contribute to more accurately defining the alterations associated with the development of HCC and thus facilitate prognosis and diagnosis of this disease.

The invention provides compositions and methods designed to detect the expression levels of those genes and proteins which may be differentially expressed depending on the state of the cell (e.g. non-cancerous vs cancerous). These expression profiles provide useful molecular tools for, amongst other applications, diagnosing the disease (HCC), or evaluating a subject's predisposition or risk of developing said disease, monitoring said disease, identifying potentially useful drugs for the treatment of said disease, as well as the toxicity and metabolism of said drugs. Changes in the expression profile of said genes and/or proteins as compared to the baseline expression (basal expression level) of said genes and/or proteins in a control sample (reference, normal or control values) may be used as an indication of such effects. Those skilled in the art may use any amongst the variety of known techniques designed to evaluate the expression levels of one or more of the genes and/or fragments thereof and/or proteins identified in the present invention in order to observe changes in the expression profile in a tissue or sample of interest.

In an aspect, the invention identifies some genes that are differentially expressed in cancerous (HCC) and non-cancerous liver tissue. Specifically, the present invention is based on the discovery that the expression level of genes ADH, AOP2, SMP30, NSLT, SDH, SAP, PGM, P4H, APOA1, CAIII, AdDHm, OAT and/or OCT, and/or the corresponding proteins (ADH, AOP2, SMP30, NSLT, SDH, SAP, PGM, P4H, APOA1, CAIII, AdDHm, OAT and/or OCT) is reduced in cancerous liver tissue samples from patients diagnosed with HCC with respect to the level of the same genes and/or proteins in liver tissue samples from subjects not diagnosed with HCC or without a clinical history of HCC (control subjects).

A reduction in the expression levels of said genes and/or proteins between the liver tissue sample from a subject under study and a liver tissue sample used as a control of, at least, 5%, advantageously of, at least, 10%, preferably of, at least, 15%, more preferably of, at least, 20%, even more preferably of, at least, 25%, is indicative that the subject whose sample has been analysed suffers from HCC, or has a risk or predisposition to develop HCC.

A person skilled in the art may select one or more of said genes and/or proteins in order to assay a particular sample. As an illustration, one may select 1, 2, 3, 4, 5, 6, 7, 8, 9, 10, 11, 12 or 13 of said genes or proteins in order to study a liver tissue sample from a subject. In a particular embodiment, 1, 2, 3, 4, 5, 6, 7, 8, 9, 10, 11 or 12 of said genes or proteins are selected. In another particular embodiment, the 13 genes or proteins are selected in order to study the liver tissue sample from the subject under study. The differential expression (reduced versus control) of said 13 genes (ADH, AOP2, SMP30, NSLT, SDH, SAP, PGM, P4H, APOA1, CAIII, AdDHm, OAT and OCT) constitutes a genomic imprint (fingerprint) of HCC. Similarly, the differential expression (reduced versus control) of said 13 proteins (ADH, AOP2, SMP30, NSLT, SDH, SAP, PGM, P4H, APOA1, CAIII, AdDHm, OAT and OCT) constitutes a proteomic imprint (fingerprint) of HCC. The identification of a panel of markers, more than the use of individual markers, might contribute to perform a more specific diagnosis of the pathology. Having a panel of markers available makes it possible, moreover, to evaluate their evolution in time and, in this way, associate the progression of the disease (or the stage of evolution thereof) with the appearance of certain representative markers at a given time.

Consequently, the evaluation and comparison of the expression levels of said genes, and/or their corresponding proteins, in a liver tissue sample from a subject may be used for purposes of HCC diagnosis or prognosis. As an illustration, a reduced level of one or more of the HCC markers identified in the present invention in a liver tissue sample from a subject with respect to the levels of the same markers in biological samples from control subjects (i.e. subjects without a clinical history of HCC and/or who do not suffer from HCC) or with normal reference values (in general, obtained from control subjects) is indicative of HCC, or of a subject's higher risk or predisposition to develop said disease. The comparison of the levels of said markers in a subject, whether or not diagnosed with HCC, at a given time with those of previous samples from the same subject may be indicative of the evolution and prognosis of said disease or of the effectiveness of the treatment which is being administered to the subject (if applicable).

Therefore, the teachings of the present invention may be used, amongst other applications, in diagnostic trials or assays or trials for the evaluation of a subject's risk or predisposition to develop HCC, in prognosis trials, in follow-up trials of the effect of the therapy administered to the subject in order to analyse the effectiveness of the therapy and the evolution of the disease, and in screening trials for potentially useful compounds in the treatment of HCC.

The invention provides, amongst other things, methods designed to detect and quantify the expression of genes ADH, AOP2, SMP30, NSLT, SDH, SAP, PGM, P4H, APOA1, CAIII, AdDHm, OAT and/or OCT, and/or that of their corresponding proteins (ADH, AOP2, SMP30, NSLT, SDH, SAP, PGM, P4H, APOA1, CAIII, AdDHm, OAT and/or OCT), in a liver tissue sample from a subject.

Therefore, in an aspect, the invention relates to an in vitro method to diagnose hepatocellular carcinoma (HCC) in a subject, or to evaluate a subject's predisposition to develop HCC, or to evaluate the progression of said pathology in a subject, or to determine the stage or severity of said disease in a subject, hereinafter, method of the invention, which comprises:
a) quantifying the expression level of a gene in a liver tissue sample from said subject, wherein said gene is selected from the gene that encodes alcohol dehydrogenase (ADH) [ADH gene], the gene that encodes antioxidant protein 2 (AOP2) [gene AOP2], the gene that encodes regucalcin (SMP30) [SMP30 gene], the gene that encodes sterol-carrying protein 2 (NSLT) [NSLT gene], the gene that encodes sorbitol dehydrogenase (SDH) [SDH gene], the gene that encodes albumin (SAP) [SAP gene], the gene that encodes phosphoglucomutase (PGM) [PGM gene], the gene that encodes phenylalanine 4-hydroxylase (P4H) [P4H gene], the gene that encodes apolipoprotein A1 (APOA1) [APOA1 gene], the gene that encodes carbonic anhydrase III (CAIII) [CAIII gene], the gene that encodes class-2 aldehyde dehydrogenase (AdDHm) [30AdDHm gene], the gene that encodes ornithine aminotransferase (OAT) [OAT gene], the gene that encodes ornithine carbamoyltransferase (OCT) [OCT gene] and combinations thereof, and
b) comparing said level to that of a control sample;
   wherein a reduction in said level with respect to the expression level in the control sample is indicative of HCC;
   or, alternatively,
a) quantifying the level of a protein in a liver tissue sample from said subject, wherein said protein is selected from alcohol dehydrogenase (ADH), antioxidant protein 2 (AOP2), regucalcin (SMP30), sterol-carrying protein 2 (NSLT), sorbitol dehydrogenase (SDH), albumin (SAP), phosphoglucomutase (PGM), phenylalanine 4-hydroxylase (P4H), apolipoprotein A1 (APOA1), carbonic anhydrase III (CAIII), class-2 aldehyde dehydrogenase (AdDHm), ornithine aminotransferase (OAT), ornithine carbamoyltransferase (OCT) and combinations thereof, and
b) comparing said level to that of a control sample;
   wherein a reduction in said level with respect to the level in the control sample is indicative of HCC.

The method provided by this invention exhibits high sensitivity and specificity, and is based on the fact that the (cancerous) liver tissue from subjects diagnosed with HCC exhibits low levels of mRNA corresponding to genes ADH, AOP2, SMP30, NSLT, SDH, SAP, PGM, P4H, APOA1, CAIII, AdDHm, OAT and/or OCT, and/or reduced concentrations of proteins ADH, AOP2, SMP30, NSLT, SDH, SAP, PGM, P4H, APOA1, CAIII, AdDHm, OAT and/or OCT, as compared to the levels of said genes and/or proteins in liver tissue samples from healthy subjects, without liver disease and without a clinical history of HCC, used as control samples.

In order to implement the method of the invention, a liver tissue sample from the subject to be studied is obtained. Said sample may be obtained by any conventional method, for example, by means of liver tissue biopsies obtained by surgical resection. The samples may be obtained from subjects previously diagnosed with HCC (patients), or from subjects who have not been previously diagnosed with HCC, or from patients diagnosed with HCC who are undergoing treatment, or from subjects diagnosed with HCC who have been previously treated.

In a particular embodiment, the method of the invention comprises quantifying the expression level of a gene selected from genes ADH, AOP2, SMP30, NSLT, SDH, SAP, PGM, P4H, APOA1, CAIII, AdDHm, OAT, OCT, and combinations thereof, in a liver tissue sample from a subject and comparing said level to that of a control liver tissue sample, wherein a reduction in said expression level with respect to the expression level of the same genes in the control sample is indicative of HCC. The method of the invention includes the possibility of quantifying not only the expression level of just one of said genes, but the possibility of quantifying the expression levels of any 2, 3, 4, 5, 6, 7, 8, 9, 10, 11 or 12 genes of said genes, and even the expression level of said 13 genes.

The expression level of a gene may be quantified by quantifying the level of messenger RNA (mRNA) of a gene, resulting from the transcription of said gene, or, alternatively, of the level of complementary DNA (cDNA) to said mRNA. In this case, the method of the invention includes performing an extraction step in order to obtain the total RNA, which may be performed by conventional techniques. Practically any conventional method may be used within the scope of the invention in order to detect and quantify the mRNA levels of genes ADH, AOP2, SMP30, NSLT, SDH, SAP, PGM, P4H, APOA1, CAIII, AdDHm, OAT and/or OCT, or of their corresponding cDNAs. As an illustration, but not limited thereto, the mRNA levels of said genes may be quantified using conventional methods, for example, methods which comprise amplification of mRNA and quantification of the amplification product of said mRNA by means of, for example, hybridisation with an appropriate labelled probe and detection of the signal or, alternatively, by means of Northern blot and the use of specific probes for the mRNA of interest (ADH, AOP2, SMP30, NSLT, SDH, SAP, PGM, P4H, APOA1, CAIII, AdDHm, OAT or OCT), mapping with S1 nuclease, RT-LCR, hybridisation, microarrays, etc. Similarly, the levels of the cDNAs corresponding to said mRNA of genes ADH, AOP2, SMP30, NSLT, SDH, SAP, PGM, P4H, APOA1, CAIII, AdDHm, OAT and/or OCT may also be quantified using conventional techniques; in this case, the method of the invention includes a cDNA synthesis step by reverse transcription (RT) of the corresponding mRNA, followed by amplification and quantification of the amplification product of said cDNA; in a particular embodiment, the amplification is conducted either qualitatively or quantitatively, by means of PCR (e.g. quantitative PCR, real-time quantitative PCR using an appropriate label, etc.), using primer oligonucleotides that specifically amplify regions of genes ADH, AOP2, SMP30, NSLT, SDH, SAP, PGM, P4H, APOA1, CAIII, AdDHm, OAT or OCT.

In another particular embodiment, the method of the invention comprises the quantification of the level (concentration) of a protein selected from proteins ADH, AOP2, SMP30, NSLT, SDH, SAP, PGM, P4H, APOA1, CAIII, AdDHm, OAT, OCT, and combinations thereof, in a liver tissue sample from a subject and comparison of said level to that of a control liver tissue sample, wherein a reduction in said level with respect to the level of the same proteins in the control sample is indicative of HCC. The method of the invention includes the possibility of quantifying not only the level of just one of said proteins, but the possibility of quantifying the levels of any 2, 3, 4, 5, 6, 7, 8, 9, 10, 11 or 12 of said proteins, and even the level of said 13 proteins.

The level (concentration) of said proteins ADH, AOP2, SMP30, NSLT, SDH, SAP, PGM, P4H, APOA1, CAIII, AdDHm, OAT and/or OCT may be quantified by any conventional method that makes it possible to detect and quantify said proteins in a sample from a subject. In this case, the method of the invention includes performing an extraction step in order to obtain a protein extract that contains said proteins, which may be performed using conventional techniques.

Practically any conventional method may be used within the scope of the invention in order to detect and quantify the levels of ADH, AOP2, SMP30, NSLT, SDH, SAP, PGM, P4H, APOA1, CAIII, AdDHm, OAT and/or OCT. As an illustration, but not limited thereto, the levels of said proteins may be quantified using conventional methods, for example, the use of antibodies with the capacity to bind to ADH, AOP2, SMP30, NSLT, SDH, SAP, PGM, P4H, APOA1, CAIII, AdDHm, OAT or OCT (or to fragments thereof which contain antigenic determinants) and subsequent quantification of the complexes formed. The antibodies used in these assays may be labelled or unlabelled. Illustrative examples of the labels which may be used include radioactive isotopes, enzymes, fluorophores, chemoluminescent reagents, enzyme substrates or co-factors, enzyme inhibitors, particles, stains, etc. There is a wide range of known assays that may be used in this invention, which use unlabelled antibodies (primary antibody) and labelled antibodies (secondary antibody); these techniques include Western-blot or Western transfer, ELISA (enzyme-linked immunosorbent assay), RIA (radioimmunoassay), competitive EIA (competitive enzyme immunoassay), DAS-ELISA (double antibody sandwich ELISA), immunocytochemical and immunohistochemical techniques, techniques based on the use of protein biochips or microarrays which include specific antibodies or assays based on colloidal precipitation in formats such as dipsticks. Other methods for detecting and quantifying said proteins ADH, AOP2, SMP30, NSLT, SDH, SAP, PGM, P4H, APOA1, CAIII, AdDHm, OAT or OCT include affinity chromatography techniques, ligand-binding assays, etc.

In a particular embodiment, the quantification of the levels of ADH, AOP2, SMP30, NSLT, SDH, SAP, PGM, P4H, APOA1, CAIII, AdDHm, OAT and/or OCT is performed by means of using of antibodies with the capacity to bind to ADH, AOP2, SMP30, NSLT, SDH, SAP, PGM, P4H, APOA1, CAIII, AdDHm, OAT or OCT (or fragments thereof which contain antigenic determinants) and subsequent quantification of the complexes formed, for example, by means of immunochemical techniques that allow for the quantification of the antigen-antibody bond, for example, Western blot, ELISA, protein biochips, etc.; preferably, the quantification of the levels of ADH, AOP2, SMP30, NSLT, SDH, SAP, PGM, P4H, APOA1, CAIII, AdDHm, OAT and/or OCT is performed by means of protein biochips or by Western blot using the appropriate antibodies that are capable of binding to said proteins. Said antibodies are available in the market or may be obtained by conventional methods known by those skilled in the art.

The method of the invention also comprises the step wherein the expression levels of genes ADH, AOP2, SMP30, NSLT, SDH, SAP, PGM, P4H, APOA1, CAIII, AdDHm, OAT and/or OCT, and/or the levels of proteins ADH, AOP2, SMP30, NSLT, SDH, SAP, PGM, P4H, APOA1, CAIII, AdDHm, OAT and/or OCT, determined in the liver tissue sample from the subject under study, are compared to the levels of said genes and/or proteins in a liver tissue sample used as a control (i.e. with the reference values generally being obtained from healthy subjects, without liver disease). The expression levels of genes ADH, AOP2, SMP30, NSLT, SDH, SAP, PGM, P4H, APOA1, CAIII, AdDHm, OAT and/or OCT, as well as the levels of proteins ADH, AOP2, SMP30, NSLT, SDH, SAP, PGM, P4H, APOA1, CAIII, AdDHm, OAT and/or OCT, may be determined by means of the above-mentioned techniques in liver tissue samples from, preferably, healthy subjects, without liver disease. A reduction in the expression levels of genes ADH, AOP2, SMP30, NSLT, SDH, SAP, PGM, P4H, APOA1, CAIII, AdDHm, OAT and/or OCT, or in the levels of proteins ADH, AOP2, SMP30, NSLT, SDH, SAP, PGM, P4H, APOA1, CAIII, AdDHm, OAT and/or OCT, in the liver tissue sample of the subject under study with respect to the corresponding levels in the control liver tissue sample is indicative of HCC.

In another aspect, the invention relates to an in vitro method for the identification and evaluation of the effectiveness of HCC treatments. The method contemplates the quantification of the levels of proteins ADH, AOP2, SMP30, NSLT, SDH, SAP, PGM, P4H, APOA1, CAIII, AdDHm, OAT and/or OCT and/or the expression levels of genes ADH, AOP2, SMP30, NSLT, SDH, SAP, PGM, P4H, APOA1, CAIII, AdDHm, OAT and/or OCT in the same subject throughout the different phases or stages of the disease, or during the periods of treatment and absence thereof, and comparison thereof to control values which are considered to be normal or with previous values for the same subject. When a therapeutic agent increases the expression of genes ADH, AOP2, SMP30, NSLT, SDH, SAP, PGM, P4H, APOA1, CAIII, AdDHm, OAT and/or OCT, or of proteins ADH, AOP2, SMP30, NSLT, SDH, SAP, PGM, P4H, APOA1, CAIII, AdDHm, OAT and/or OCT, said agent becomes a candidate for the treatment of HCC.

In a particular embodiment, the invention relates to an in vitro method for evaluating the effect of the therapy administered to a subject with HCC, which comprises (i) quantifying the expression level of a gene in a liver tissue sample from said subject, wherein said gene is selected from genes ADH, AOP2, SMP30, NSLT, SDH, SAP, PGM, P4H, APOA1, CAIII, AdDHm, OAT, OCT and combinations thereof, and/or the level of a protein selected from ADH, AOP2, SMP30, NSLT, SDH, SAP, PGM, P4H, APOA1, CAIII, AdDHm, OAT, OCT, and combinations thereof, and (ii) comparing said level to that of a liver tissue sample from the same subject, prior to administration of the therapy. In this case, an increase in the expression level of said genes and/or proteins in relation to the levels prior to administration of the therapy is indicative that the therapy administered to the subject is effective. The quantification of the expression levels of said genes and/or proteins and the comparison of said expression levels may be performed by conventional methods, as has been previously described in relation to the method of the invention.

The nucleotide sequences of genes ADH, AOP2, SMP30, NSLT, SDH, SAP, PGM, P4H, APOA1, CAIII, AdDHm, OAT and/or OCT may be used to design primer oligonucleotides which are useful for amplifying specific fragments of said genes, as well as to obtain potentially useful probes in identifying said genes; therefore, said nucleotide sequences of said genes, as well as said primer oligonucleotides and probes may be used to diagnose HCC in vitro in a subject or to evaluate a subject's predisposition to develop HCC, or to evaluate the progression of said disease in a subject, or to determine the stage or severity of said disease in a subject, or to identify and evaluate the effectiveness of an HCC treatment, for example, to evaluate the effect of the therapy administered to a subject suffering from said disease.

Similarly, proteins ADH, AOP2, SMP30, NSLT, SDH, SAP, PGM, P4H, APOA1, CAIII, AdDHm, OAT and/or OCT, or fragments thereof which comprise one or more antigenic determinants (epitopes) of said proteins, may be used to produce antibodies against said proteins. Therefore, said proteins and fragments thereof, as well as said antibodies with the capacity to bind to said proteins or fragments thereof, may be used to diagnose HCC in vitro in a subject or to evaluate a subject's predisposition to develop HCC, or to evaluate the progression of said disease in a subject, or to determine the stage or severity of said disease in a subject, or to identify and evaluate the effectiveness of an HCC treatment, for example, to evaluate the effect of the therapy administered to a subject suffering from said disease.

In another aspect, the invention relates to the use of an antibody to diagnose HCC in vitro in a subject or to evaluate a subject's predisposition to develop HCC, or to evaluate the progression of said disease in a subject, or to determine the stage or severity of said disease in a subject, or to identify and evaluate the effectiveness of an HCC treatment, for example, to evaluate the effect of the therapy administered to a subject suffering from said disease, wherein said antibody is an antibody which has the capacity to bind to a protein selected from ADH, AOP2, SMP30, NSLT, SDH, SAP, PGM, P4H, APOA1, CAIII, AdDHm, OAT and OCT, or to fragments thereof containing antigenic determinants of said proteins. Said antibodies may be recombinant antibodies, monoclonal antibodies, polyclonal antibodies, intact, or fragments thereof which preserve the capacity to bind to said proteins (ADH, AOP2, SMP30, NSLT, SDH, SAP, PGM, P4H, APOA1, CAIII, AdDHm, OAT or OCT), for example, Fab, scFv, etc. fragments, human, humanised or non-human antibodies.

The molecular biomarkers identified in the present invention, such as genes ADH, AOP2, SMP30, NSLT, SDH, SAP, PGM, P4H, APOA1, CAIII, AdDHm, OAT and/or OCT, and/or proteins ADH, AOP2, SMP30, NSLT, SDH, SAP, PGM, P4H, APOA1, CAIII, AdDHm, OAT and/or OCT, may be used in the screening, search, identification, development and evaluation of the effectiveness of compounds in the treatment of HCC.

Therefore, in another aspect, the invention relates to a method for the screening, search, identification, development and evaluation of the effectiveness of compounds in the treatment of HCC, which comprises (i) contacting a cellular system which expresses a reduced level of a gene with the candidate compound, and (ii) evaluating the expression of said gene, such that, if the expression of said gene increases, the candidate compound is a potentially useful compound in the treatment of HCC, wherein said gene is selected from genes ADH, AOP2, SMP30, NSLT, SDH, SAP, PGM, P4H, APOA1, CAIII, AdDHm, OAT, OCT and combinations thereof. Said cellular system may be practically any cellular system wherein one or more of said markers are reduced, e.g. transformed human cell lines which exhibit a decrease in the level of such markers, such as the HepG2, Huh7, etc. cell lines. The expression of said genes may be evaluated by any conventional method, for example, by quantification of the levels of mRNA of said genes or their corresponding cDNAs, or by quantification of said proteins ADH, AOP2, SMP30, NSLT, SDH, SAP, PGM, P4H, APOA1, CAIII, AdDHm, OAT or OCT by any of the methods previously mentioned in relation to the method of the invention. When a compound increases the expression levels of any of the genes ADH, AOP2, SMP30, NSLT, SDH, SAP, PGM, P4H, APOA1, CAIII, AdDHm, OAT and/or OCT or the levels (concentration) of proteins ADH, AOP2, SMP30, NSLT, SDH, SAP, PGM, P4H, APOA1, CAIII, AdDHm, OAT and/or OCT, said compound becomes a potentially useful candidate for the treatment of HCC. In another aspect, the invention relates to the use of said genes ADH, AOP2, SMP30, NSLT, SDH, SAP, PGM, P4H, APOA1, CAIII, AdDHm, OAT and/or OCT, and/or of said proteins ADH, AOP2, SMP30, NSLT, SDH, SAP, PGM, P4H, APOA1, CAIII, AdDHm, OAT and/or OCT, in a method for the screening, search, identification, development and evaluation of the effectiveness of compounds in the treatment of HCC.

Those compounds which enhance the expression of genes ADH, AOP2, SMP30, NSLT, SDH, SAP, PGM, P4H, APOA1, CAIII, AdDHm, OAT and/or OCT, or the activity of proteins ADH, AOP2, SMP30, NSLT, SDH, SAP, PGM, P4H, APOA1, CAIII, AdDHm, OAT and/or OCT, may be used in the treatment of HCC. Illustrative examples, not limited thereto, of said compounds include positive modulators of the expression of said genes or the activity of said proteins.

In another aspect, the invention relates to a pharmaceutical composition which comprises a therapeutically effective amount of a compound that enhances the expression of genes ADH, AOP2, SMP30, NSLT, SDH, SAP, PGM, P4H, APOA1, CAIII, AdDHm, OAT and/or OCT, or the activity of proteins ADH, AOP2, SMP30, NSLT, SDH, SAP, PGM, P4H, APOA1, CAIII, AdDHm, OAT and/or OCT, together with one or more pharmaceutically acceptable excipients and/or carriers. In a particular embodiment, said pharmaceutical composition comprises a positive modulator of the expression of said genes or a positive modulator of the activity of said proteins.

The excipients, carriers and auxiliary substances must be pharmaceutically and pharmacologically tolerable and must be able to be combined with other components of the formulation and not exert any adverse effect on the subject under treatment. The pharmaceutical compositions may be offered in any appropriate form of administration, for example, in pharmaceutical forms suitable for oral and parenteral administration (including, e.g. intravenous, subcutaneous, intradermal, intramuscular, intraperitoneal and intrathecal administration). The formulations may be single-dose, and shall be prepared in accordance with classical galenic methods. A review of the different pharmaceutical forms for drug administration and the preparation thereof may be found in the book “Tratado de Farmacia Galénica”, by C. Faulí i Trillo, 10th Edition, 1993, Luzán 5, S.A. de Ediciones.

In another aspect, the invention relates to the use of a compound which enhances the expression of genes ADH, AOP2, SMP30, NSLT, SDH, SAP, PGM, P4H, APOA1, CAIII, AdDHm, OAT and/or OCT, or the activity of proteins ADH, AOP2, SMP30, NSLT, SDH, SAP, PGM, P4H, APOA1, CAIII, AdDHm, OAT and/or OCT, in the manufacture of a pharmaceutical composition for the treatment of HCC.

In another aspect, the present invention relates to a pair of primer oligonucleotides which are useful in the specific amplification of a fragment of mRNA or cDNA of a gene, wherein said gene is selected from genes ADH, AOP2, SMP30, NSLT, SDH, SAP, PGM, P4H, APOA1, CAIII, AdDHm, OAT and OCT. In a particular embodiment, said pair of primer oligonucleotides is selected from the group of oligonucleotide pairs consisting of SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO: 24, and SEQ ID NO: 25 and SEQ ID NO: 26.

In another aspect, the present invention relates to a probe for identifying a gene selected from genes ADH, AOP2, SMP30, NSLT, SDH, SAP, PGM, P4H, APOA1, CAIII, AdDHm, OAT and OCT. In a particular embodiment, the invention provides 1, 2, 3, 4, 5, 6, 7, 8, 9, 10, 11, 12 or 13 probes for identifying said 13 genes, with said probes being fixed onto a solid support, such as a membrane, a plastic or a glass, optionally treated in order to facilitate fixation of said probes onto the support. Said solid support, which comprises, at least, one probe for identifying one gene, wherein said gene is selected from genes ADH, AOP2, SMP30, NSLT, SDH, SAP, PGM, P4H, APOA1, CAIII, AdDHm, OAT and OCT, may be used in the identification of the transcription products of said genes (or their corresponding cDNAs) by means of array technology and constitutes an additional aspect of this invention. In a particular embodiment, said solid support comprises 1, 2, 3, 4, 5, 6, 7, 8, 9, 10, 11, 12 or 13 of said probes.

In another aspect, the present invention relates to an antibody with the capacity to bind to a protein selected from ADH, AOP2, SMP30, NSLT, SDH, SAP, PGM, P4H, APOA1, CAIII, AdDHm, OAT and OCT. In a particular embodiment, the invention provides 1, 2, 3, 4, 5, 6, 7, 8, 9, 10, 11, 12 or 13 antibodies for the identification of said 13 proteins, said antibodies being fixed onto a solid support, such as a membrane, a plastic or a glass, optionally treated in order to facilitate fixation of said antibodies onto the support. Said solid support, which comprises, at least, one antibody with the capacity to bind to a protein and identify it, wherein said protein is selected from proteins ADH, AOP2, SMP30, NSLT, SDH, SAP, PGM, P4H, APOA1, CAIII, AdDHm, OAT and OCT, may be used in the identification of proteins by means of array technology and constitutes an additional aspect of this invention. In a particular embodiment, said solid support comprises 1, 2, 3, 4, 5, 6, 7, 8, 9, 10, 11, 12 or 13 of said antibodies.

In another aspect, the invention relates to a kit useful in the implementation of the methodology described herein. Thus, said kit may contain all or part of the necessary reagents for the detection of the expression levels of genes ADH, AOP2, SMP30, NSLT, SDH, SAP, PGM, P4H, APOA1, CAIII, AdDHm, OAT and/or OCT, or for the detection of the levels of proteins ADH, AOP2, SMP30, NSLT, SDH, SAP, PGM, P4H, APOA1, CAIII, AdDHm, OAT and/or OCT, which include the following:
one or more pairs of primer oligonucleotides for the specific amplification of fragments of the mRNAs (or of their corresponding cDNAs) of genes ADH, AOP2, SMP30, NSLT, SDH, SAP, PGM, P4H, APOA1, CAIII, AdDHm, OAT and OCT; and/or
one or more probes for the identification of one or more genes selected from genes ADH, AOP2, SMP30, NSLT, SDH, SAP, PGM, P4H, APOA1, CAIII, AdDHm, OAT and OCT; and/or
one or more antibodies with the capacity to bind to a protein selected from ADH, AOP2, SMP30, NSLT, SDH, SAP, PGM, P4H, APOA1, CAIII, AdDHm, OAT and OCT, or to fragments thereof which contain antigenic determinants.

That kit may contain 1, 2, 3, 4, 5, 6, 7, 8, 9, 10, 11, 12 or 13 pairs of primer oligonucleotides, each of them being specific for one of said genes. Illustrative, non limitative examples of pairs of primer oligonucleotides which can be included in said kit include the following oligonucleotide pairs: SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO: 24, and/or SEQ ID NO: 25 and SEQ ID NO: 26.

Alternatively, said kit may contain 1, 2, 3, 4, 5, 6, 7, 8, 9, 10, 11, 12 or 13 probes, each of them being specific for the identification of one of said genes. In another alternative embodiment, said kit may contain 1, 2, 3, 4, 5, 6, 7, 8, 9, 10, 11, 12 or 13 antibodies, each of them having the capacity to bind to one of said proteins.

Said reagents, specifically, the probes and the antibodies, may be fixed onto a solid support, as previously mentioned.

Said kit may be used to diagnose HCC in vitro in a subject, or to evaluate a subject's predisposition to develop HCC, or to evaluate the progression of said disease in a subject, or to determine the stage or severity of said disease in a subject, or to identify and evaluate the effectiveness of an HCC treatment, for example, to evaluate the effect of the therapy administered to a subject suffering from said disease.

### Markers of Stages Prior to the Development of HCC

As previously indicated, the invention also identifies some genes which are differentially expressed in cirrhotic liver tissue (i.e. at a stage prior to the development of HCC) and healthy liver tissue; specifically, it has also been observed that the expression level of genes ADH, AOP2, SMP30, NSLT, SDH, SAP and/or PGM, and/or of the corresponding proteins (ADH, AOP2, SMP30, NSLT, SDH, SAP and/or PGM), is reduced in liver tissue samples from patients with cirrhosis (stage prior to the development of HCC) with respect to the level of the same genes and/or proteins in liver tissue samples from healthy subjects, without liver disease (control subjects). A reduction in the expression levels of said genes and/or proteins between the liver tissue sample from a subject under study and a liver tissue sample used as a control of, at least, 5%, advantageously of, at least, 10%, preferably of, at least, 15%, more preferably of, at least, 20%, even more preferably of, at least, 25%, is indicative that the subject under study suffers from cirrhosis which, in time, may evolve to HCC.

A person skilled in the art may select one or more of said genes and/or proteins in order to assay a specific sample. As an illustration, 1, 2, 3, 4, 5, 6 or 7 of said genes or proteins may be selected in order to study a liver tissue sample from a subject. In a particular embodiment, 1, 2, 3, 4, 5 or 6 of said genes or proteins are selected. In another particular embodiment, the 7 genes or proteins are selected in order to study the liver tissue sample of the subject under study. The differential expression (reduced versus control) of said 7 genes (ADH, AOP2, SMP30, NSLT, SDH, SAP and PGM) constitutes a genomic imprint (fingerprint) of a stage prior to the development of HCC (cirrhosis). Similarly, the differential expression (reduced versus control) of said 7 proteins (ADH, AOP2, SMP30, NSLT, SDH, SAP and PGM) constitutes a proteomic imprint (fingerprint) of a stage prior to the development of HCC (cirrhosis).

Consequently, the evaluation and comparison of the expression levels of said genes and/or their corresponding proteins, in a liver tissue sample from a subject, may be used to identify a stage prior to the development of HCC and take the appropriate measures.

Therefore, in another aspect, the invention relates to an in vitro method to identify a stage prior to the development of hepatocellular carcinoma (HCC) in a subject, which comprises:
a) quantifying the expression level of a gene in a liver tissue sample from said subject, wherein said gene is selected from genes ADH, AOP2, SMP30, NSLT, SDH, SAP, PGM, and combinations thereof, and
b) comparing said level to that of a control sample;
   wherein a reduction in said level with respect to the expression level in the control sample is indicative of a stage prior to the development of HCC; or, alternatively,
a) quantifying the level of a protein in a liver tissue sample from said subject, wherein said protein is selected from ADH, AOP2, SMP30, NSLT, SDH, SAP, PGM, and combinations thereof, and
b) comparing said level to that of a control sample;
   wherein a reduction in said level with respect to the level in the control sample is indicative of a stage prior to the development of HCC.

For the implementation of said method, a liver tissue sample from the subject to be studied is obtained, for example, by means of a biopsy, as it has been previously indicated in relation to the in vitro method designed to diagnose HCC provided by this invention. The samples may be obtained from subjects with cirrhosis.

In a particular embodiment, the previously described method comprises quantifying the expression level of a gene selected from genes ADH, AOP2, SMP30, NSLT, SDH, SAP, PGM, and combinations thereof, in a liver tissue sample from a subject and comparing said expression level to a control liver tissue sample, wherein a reduction in said expression level with respect to the expression level of the same genes in the control sample is indicative of a stage prior to the development of HCC. The implementation of said method includes the possibility of quantifying not only the expression level of just one of said genes, but the possibility of quantifying the expression levels of any 2, 3, 4, 5, or 6 of said genes, and even the expression level of said 7 genes. The expression level of said gene may be quantified by means of the quantification of the level of mRNA of said gene, or, alternatively, of the level of cDNA to said mRNA, as has been previously mentioned in relation to the in vitro method designed to diagnose HCC provided by this invention. In a particular embodiment, the expression level of mRNA is determined by means of Northern blot and the use of specific probes for the mRNA of interest (ADH, AOP2, SMP30, NSLT, SDH, SAP or PGM), or by means of quantitative PCR, e.g. real-time quantitative PCR, using primer oligonucleotides which specifically amplify regions of genes ADH, AOP2, SMP30, NSLT, SDH, SAP or PGM.

In another particular embodiment, the previously described method comprises quantifying the level (concentration) of a protein selected from proteins ADH, AOP2, SMP30, NSLT, SDH, SAP, PGM, and combinations thereof, in a liver tissue sample from a subject and comparing said level to that of a control liver tissue sample, wherein a reduction in said level with respect to the level of the same proteins in the control sample is indicative of a stage prior to the development of HCC. The implementation of said method includes the possibility of quantifying not only the level of just one of said proteins, but the possibility of quantifying the levels of any 2, 3, 4, 5, or 6 of said proteins, and even the level of said 7 proteins. The level of said proteins ADH, AOP2, SMP30, NSLT, SDH, SAP and/or PGM may be quantified by any conventional method, as it has been previously mentioned in relation to the in vitro method designed to diagnose HCC provided by this invention. In a particular embodiment, the quantification of the levels of ADH, AOP2, SMP30, NSLT, SDH, SAP and/or PGM is performed by means of using antibodies with the capacity to bind to said proteins (or to fragments thereof which contain antigenic determinants) and subsequent quantification of the complexes formed, for example, by means of immunochemical techniques that allow for the quantification of the antigen-antibody complex, for example, Western blot, ELISA, protein biochips, etc.

The previously described method also comprises the step of comparing the expression levels of genes ADH, AOP2, SMP30, NSLT, SDH, SAP and/or PGM, and/or the levels of proteins ADH, AOP2, SMP30, NSLT, SDH, SAP and/or PGM, determined in the liver tissue sample of the subject under study, to the levels of said genes and/or proteins in a liver tissue sample used as a control (i.e. with the reference values generally obtained from healthy subjects, without liver disease). A reduction in the expression levels of genes ADH, AOP2, SMP30, NSLT, SDH, SAP and/or PGM, or in the levels of proteins ADH, AOP2, SMP30, NSLT, SDH, SAP and/or PGM, in the liver tissue sample of the subject under study with respect to the corresponding levels in the control liver tissue sample is indicative of a stage prior to the development of HCC.

The nucleotide sequences of genes ADH, AOP2, SMP30, NSLT, SDH, SAP and/or PGM may be used to design primer oligonucleotides for amplifying specific fragments of said genes as well as to obtain probes which are potentially useful in identifying said genes; therefore, said nucleotide sequences of said genes, as well as said primer oligonucleotides and probes, may be used to in vitro identify a stage prior to the development of HCC in a subject.

Similarly, proteins ADH, AOP2, SMP30, NSLT, SDH, SAP and/or PGM, or fragments thereof which comprise one or more antigenic determinants (epitopes) of said proteins, may be used to produce antibodies against said proteins. Therefore, said proteins and fragments thereof, as well as said antibodies with the capacity to bind to said proteins, may be used to in vitro identify a stage prior to the development of HCC in a subject.

In another aspect, the invention relates to the use of an antibody to in vitro identify a stage prior to the development of HCC in a subject, wherein said antibody is an antibody with the capacity to bind to a protein selected from ADH, AOP2, SMP30, NSLT, SDH, SAP and PGM, or to fragments thereof which contain antigenic determinants of said proteins. Said antibodies may be recombinant antibodies, monoclonal antibodies, polyclonal antibodies, intact, or fragments thereof which preserve the capacity to bind to said proteins (ADH, AOP2, SMP30, NSLT, SDH, SAP or PGM), for example, Fab, scFv, etc. fragments, human, humanised or non-human antibodies.

The identified molecular biomarkers, such as genes ADH, AOP2, SMP30, NSLT, SDH, SAP and/or PGM, and/or proteins ADH, AOP2, SMP30, NSLT, SDH, SAP and/or PGM, may be used in the screening, search, identification, development and evaluation of the effectiveness of compounds in the treatment of stages prior to the development of HCC in a subject.

Therefore, in another aspect, the invention relates to a method for the screening, search, identification, development and evaluation of the effectiveness of compounds in the treatment of stages prior to the development of HCC in a subject, which comprises (i) contacting a cellular system that expresses a reduced level of a gene with the candidate compound, and ii) evaluating the expression of said gene, such that if the expression of said gene increases, the candidate compound is a potentially useful compound in the treatment of a stage prior to the development of HCC, wherein said gene is selected from genes ADH, AOP2, SMP30, NSLT, SDH, SAP, PGM and combinations thereof. Said cellular system may be practically any cellular system wherein one or more of said markers are reduced, e.g. transformed human cell lines which exhibit a decrease in the level of such markers. The expression of said genes may be evaluated by any conventional method, for example, by quantification of the levels of mRNA pertaining to said genes or their corresponding cDNAs, or by quantification of said proteins by any of the above-mentioned methods. When a compound increases the expression of ADH, AOP2, SMP30, NSLT, SDH, SAP and/or PGM, said compound becomes a potentially useful candidate for the treatment of a stage prior to the development of HCC.

Consequently, in another aspect, the invention relates to the use of said genes ADH, AOP2, SMP30, NSLT, SDH, SAP and/or PGM, and/or of said proteins ADH, AOP2, SMP30, NSLT, SDH, SAP and/or PGM, in a method for the screening, search, identification, development and evaluation of the effectiveness of compounds in the treatment of stages prior to the development of HCC in a subject.

Those compounds which enhance the expression of genes ADH, AOP2, SMP30, NSLT, SDH, SAP and/or PGM, or the activity of proteins ADH, AOP2, SMP30, NSLT, SDH, SAP and/or PGM may be used in the treatment of stages prior to the development of HCC. Illustrative examples, not limited thereto, of said compounds include positive modulators of the expression of said genes or of the activity of said proteins.

In another aspect, the invention relates to a pharmaceutical composition which comprises a therapeutically effective amount of a compound which enhances the expression of genes ADH, AOP2, SMP30, NSLT, SDH, SAP and/or PGM, or the activity of proteins ADH, AOP2, SMP30, NSLT, SDH, SAP and/or PGM, together with one or more pharmaceutically acceptable excipients and/or carriers. In a particular embodiment, said pharmaceutical composition comprises a positive modulator of the expression of said genes or a positive modulator of the activity of said proteins.

The excipients, carriers and auxiliary substances must be pharmaceutically and pharmacologically tolerable and must be able to be combined with other components of the formulation and not exert any adverse effect on the subject under treatment. The pharmaceutical compositions may be offered in any appropriate form of administration, for example, in pharmaceutical forms suitable for oral and parenteral administration (including, e.g. intravenous, subcutaneous, intradermal, intramuscular, intraperitoneal and intrathecal administration). The formulations may be single-dose, and shall be prepared in accordance with classical galenic methods. A review of the different pharmaceutical forms for drug administration and the preparation thereof may be found in the book “Tratado de Farmacia Galénica”, by C. Faulí i Trillo, 10th Edition, 1993, Luzán 5, S.A. de Ediciones.

In another aspect, the invention relates to the use of a compound which enhances the expression of genes ADH, AOP2, SMP30, NSLT, SDH, SAP and/or PGM, or the activity of proteins ADH, AOP2, SMP30, NSLT, SDH, SAP and/or PGM, in the manufacture of a pharmaceutical composition for the treatment of a stage prior to the development of HCC.

In another aspect, the present invention relates to a pair of primer oligonucleotides for specifically amplifying a fragment of mRNA or cDNA of a gene, wherein said gene is selected from genes ADH, AOP2, SMP30, NSLT, SDH, SAP and/or PGM. In a particular embodiment, said pair of primer oligonucleotides is selected from the group of oligonucleotide pairs consisting of SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, and SEQ ID NO: 13 and SEQ ID NO: 14.

In another aspect, the present invention relates to a probe for identifying a gene selected from genes ADH, AOP2, SMP30, NSLT, SDH, SAP and PGM. In a particular embodiment, the invention provides 1, 2, 3, 4, 5, 6 or 7 probes for identifying said 7 genes, said probes being fixed onto a solid support, such as a membrane, a plastic or a glass, optionally treated in order to facilitate fixation of said probes onto the support. Said solid support, which comprises, at least, one probe for identifying one gene, wherein said gene is selected from genes ADH, AOP2, SMP30, NSLT, SDH, SAP and/or PGM, may be used in the identification of the transcription products of said genes (or of their corresponding cDNAs) by means of array technology and constitutes an additional aspect of this invention. In a particular embodiment, said solid support comprises 1, 2, 3, 4, 5, 6 or 7 of said probes.

In another aspect, the present invention relates to an antibody with the capacity to bind to a protein selected from ADH, AOP2, SMP30, NSLT, SDH, SAP and PGM. In a particular embodiment, the invention provides 1, 2, 3, 4, 5, 6 or 7 antibodies for identifying said 7 proteins, said antibodies being fixed onto a solid support, such as a membrane, a plastic or a glass, optionally treated in order to facilitate fixation of said antibodies to the support. Said solid support, which comprises, at least, one antibody with the capacity to bind to a protein and identify it, wherein said protein is selected from proteins ADH, AOP2, SMP30, NSLT, SDH, SAP and PGM, may be used in the identification of proteins by means of array technology and constitutes an additional aspect of this invention. In a particular embodiment, said solid support comprises 1, 2, 3, 4, 5, 6 or 7 of said antibodies.

In another aspect, the invention relates to a kit useful in implementing an in vitro method designed to identify a stage prior to the development of HCC, previously described. Said kit may contain all or part of the necessary reagents to detect the expression levels of genes ADH, AOP2, SMP30, NSLT, SDH, SAP and/or PGM, or of proteins ADH, AOP2, SMP30, NSLT, SDH, SAP and/or PGM, which include the following:
one or more pairs of primer oligonucleotides for specifically amplifying fragments of the mRNAs (or of their corresponding cDNAs) of genes ADH, AOP2, SMP30, NSLT, SDH, SAP or PGM; and/or
one or more probes for identifying one or more genes selected from genes ADH, AOP2, SMP30, NSLT, SDH, SAP and PGM; and/or
one or more antibodies with the capacity to bind to a protein selected from ADH, AOP2, SMP30, NSLT, SDH, SAP and PGM, or to fragments thereof which contain antigenic determinants.

That kit may contain 1, 2, 3, 4, 5, 6 or 7 pairs of primer oligonucleotides, each of them being specific for one of said genes. Illustrative, non limitative examples of pairs of primer oligonucleotides which can be included in said kit include the following oligonucleotide pairs: SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, and/or SEQ ID NO: 13 and SEQ ID NO: 14.

Alternatively, said kit may contain 1, 2, 3, 4, 5, 6 or 7 probes, each of them being specific for the identification of one of said genes. In another alternative embodiment, said kit may contain 1, 2, 3, 4, 5, 6 or 7 antibodies, each of them having the capacity to bind to one of said proteins, or to fragments thereof which contain antigenic determinants.

Said reagents, specifically, the probes and the antibodies, may be fixed onto a solid support, as previously mentioned.

The following example illustrates the invention, although it should not be considered to limit the scope thereof.

### Example 1

### Material and Methods

### Laboratory Animals

MAT1A−/− mice raised in inventors' own colony and developed at inventors' laboratory (Lu S C, Alvarez L, Huang Z Z, Chen L, An W, Corrales F J, Avila M A, Kanel G, Mato J M. Methionine Adenosyltransferase 1A Knockout Mice Are Predisposed to Liver Injury and Exhibit Increased Expression of Genes Involved in Proliferation. Proc Natl Acad Sci USA 2001; 98: 5560-65) have been used. Following sacrifice of the Wild-Type (WT) and MAT1A−/− mice and extraction of the liver thereof, the size of the tumours present in the MAT1A−/− liver have been measured. The liver samples were immediately frozen in liquid nitrogen and kept at −80° C. All the experimental procedures were conducted in accordance with the institutional guidelines of the University of Navarra regarding the use of and work with laboratory animals.

### Patients

Liver samples from several groups have been obtained:

a) Samples from control individuals (n=10). The control group samples have been obtained from patients who have been subject to a cholecystectomy for the treatment of asymptomatic cholelithiasis and have given their consent to be subject to a liver biopsy. These patients have exhibited normal liver histology and their liver functions have been normal.

b) Samples from patients who suffer from alcoholic cirrhosis (n=5).

c) Patients who suffer from viral cirrhosis (n=5).

d) Patients with HCC of different etiology (n=10).

e) Liver samples of the non-tumour area of patients with HCC obtained from healthy liver (n=7).

f) Liver samples from the non-tumour area of patients with HCC of different etiology: 3 from Hepatitis C and 5 from viral cirrhosis (n=8).

The pathological samples have been obtained during surgery or at the time of liver transplant.

All the tissues have been immediately frozen in liquid nitrogen in order to, subsequently, perform RNA extraction. This study has been approved by the research committee of the University of Navarra. The studies have been conducted in accordance with the ethical standards formulated in the 1975 Declaration of Helsinki (revised in 1983).

### Proteomic Analysis

Characterisation of the liver proteome and the alterations associated with the experimental model were performed by combining the resolution of the mixture of proteins from the biological samples by means of two-dimensional electrophoresis and subsequent identification of the proteins of interest by mass spectrometry according to the proteomic approach outlined below:

A) PREPARATORY PHASE
Homogenisation/Solubilisation of the proteins
Isoelectrofocusing (1^{st} D)
Reduction/alkylation
SDS-PAGE (2^{nd} D)
Staining/destaining

B) PREPARATORY PHASE
Image analysis
Differential band excision
Enzyme digestion
Identification by means of Mass Spectrometry (MS)

50 mg liver tissue pieces from both WT mice and MAT1A−/− mice were homogenised in 20 volumes of lysis buffer, which had a composition of: 7 M urea, 2 M thiourea, 4% (v/v) 3-[(3-chloramidopropyl)dimethyl-ammonio]-1-propanesulfonate (CHAPS), 1% (v/v) dithiotreitol (DTT) and 0.5% 3-10 ampholytes (BioRad). The extracts were centrifuged at 100,000×g for 1 hour at 15° C. The protein concentration in the supernatants obtained was measured by means of the Bradford method (BioRad) using bovine albumin as the standard.

The 1^{st} dimension was performed in isoelectrofocusing sources (Protean IEF cell by BioRad), using 17 cm gels (ReadyStrips IPG strips by BioRad) with different pH ranges. The sample (150-1,000 μg) was loaded by means of an active rehydration process for 12 hours at 50 V and 20° C. Following rehydration, the gels were subject to progressively increasing voltage, until 60,000 V× hour were reached, using the protocol recommended by the manufacturer. The strips were equilibrated in a buffer with the following composition: 50 mM Tris/HCl, pH 7.5, 6 M urea, 30% (v/v) glycerol, 2% (v/v) sodium dodecylsulphate (SDS), 2% (v/v) DTT and traces of bromophenol blue and, subsequently, were incubated in the same buffer, which contained 2.5% iodoacetamide in the absence of DTT. The strips were directly placed on 12.5% polyacrylamide gels (18 cm×20 cm×1 mm) and sealed with 1% low-melting-point agarose in order to fix their position. The SDS-PAGE gels (2^{nd} dimension) were run for 14 hours at 90 V and stained with Coomassie Safe Stain (Invitrogen). Alternatively, silver stain (Amersham Biosciences) and Sypro Ruby fluorescent stain (BioRad) were used. Those gels stained with Coomassie or with silver were digitalised in a GS-800 calibrated densitometer (Bio-Rad). Those gels stained with fluorescence were scanned with the Molecular Imager FX densitometer (Bio-Rad). The images obtained were analysed using BioRad's PDQuest 7.1 software. The only differences that were considered to be valid were those which showed a greater than 3-fold variation in expression levels and were confirmed in at least 3 independent experiments. All the samples were processed in duplicate. The bands pertaining to the differentially expressed proteins were manually extracted from the gel and automatically processed in a robot (Micromass' MassPrep station). Those bands of interest stained with Coomassie or with fluorescence were destained with 50 mM ammonium bicarbonate and 50% (v/v) acetonitrile, whilst those bands stained with silver were destained with 15 mM potassium ferricyanide and 50 mM sodium thiosulphate. The proteins were reduced with 10 mM DTT in 100 mM sodium bicarbonate and alkylated with 55 mM iodoacetamide in the same buffer. Subsequently, digestion was performed with 6 ng/μl of trypsin in 50 mM ammonium bicarbonate for 5 hours at 37° C. Alternatively, the procedure developed by Sigma was used. The peptide hydrolisate obtained was extracted with 1% (v/v) formic acid and 2% (v/v) acetonitrile. Subsequently, 1.2 μl of each digest were mixed with 1.2 μl of a saturated solution of α-cyano-4-hydroxy-trans-cynnamic acid in 0.1% trifluoroacetic acid and 50% (v/v) acetonitrile. The mixtures were deposited on plates compatible with MALDI-TOF mass spectrometry. The digests were analysed by means of mass spectrometry using Waters' MALDI-TOF GL-REF spectrometer. Sequencing and characterisation of the peptides was performed on a Waters' Q-TOF micro spectrometer. Nano-liquid-chromatography was performed using a CapLC™ system by Waters. Separation of the tryptic digest by reverse-phase chromatography was performed on an Atlantis C-18 column, 3 μm, 75 μm×10 cm, Nano Ease™ by Waters. The column was equilibrated in 5% acetonitrile and 0.2% formic acid. Following injection of 6 μl of the sample, the column was washed for 5 minutes with the same buffer, and the peptides were eluted with a linear gradient of 5-50% acetonitrile for 30 minutes with a constant flow of 0.2 μl/min. The column was connected to the mass spectrometer using a PicoTip nano-electrospray ionisation source (Waters). The capillary temperature was 80° C. and the voltage was 1.8-2.2 Kv. The fragmentation data obtained were automatically collected using the established programme. The 3 most intense ions at each retention time were fragmented by induced collision (CID) with a 2.5 window and a relative collision energy of 35%. The mass spectrometry data were processed using the Masslynx 4.0 software (Waters). For protein identification, the Proteinlynx Global Server 2.0 software (Waters) was used. The databases used for the searches were SwissProt (Swiss-Prot Release 46.3 with 176,469 entries) and Ensembl (Ensembl Mouse Release 29.33 with 31,535 entries). Finally, classification and functional interpretation of the identified proteins were performed using the GARBAN software (Luis A, Martínez-Cruz, Angel Rubio, María L. Martínez-Chantar, Alberto Labarga, Isabel Barrio, Adam Podhorski, Victor Segura, José L. Sevilla Campo, Matías A. Ávila and Jose M. Mato. GARBAN: A New Tool for Genomic Analysis and Rapid Biological Annotation of cDNA Microarray and Proteomic Data. Bioinformatics 2003; 19:2158-60).

### RNA Isolation, RT-PCR and Real-Time PCR

For the extraction of the RNA present in the human samples, Sigma's TRI reagent was used. The RNA (2 μg) has been treated with DNAse I (Invitrogen) prior to performing reverse transcription with M-MLV Reverse Transcriptase (Invitrogen) in the presence of RNAse OUT (Invitrogen). All the primer oligonucleotides used have been designed to distinguish between amplification of the genomic DNA and of the cDNA. All the PCR products have been sequenced in order to confirm the specificity. Real-time PCR has been performed with 1/20 of the RT reaction using an iCycler (Bio-Rad) and the iQ SYBR Green Supermix mixture (Bio-Rad). The following primer oligonucleotides were used:

Alcohol dehydrogenase (ADH):

(SEQ ID NO: 1)

5′-GATGCCTCTGATTGGTCTGG-3′
and

(SEQ ID NO: 2)

5′-CTTGGATGTCACAAACAGCTC-3′;
Antioxidant protein 2 (AOP2):

(SEQ ID NO: 3)

5′-CAACTTTGAGGCCAATACCAC-3′
and

(SEQ ID NO: 4)

5′-TCCTTGCTCCAGGCAAGATG-3′;
Senescence marker protein 30 (SMP30):

(SEQ ID NO: 5)

5′-TCAATGATGGGAAGGTGGATC-3′
and

(SEQ ID NO: 6)

5′-AGGTCATAGTCAAAGGCATCC-3′;
Non-specific lipid transfer protein (NSLT):

(SEQ ID NO: 7)

5′-TGGCTCTTGGGTTTGAGAAG-3′
and

(SEQ ID NO: 8)

5′-CATCTTGGAACTGGGAATACG-3′;
Sorbitol dehydrogenase (SDH):

(SEQ ID NO: 9)

5′-ATCTTCTTCTGTGCCACGCC-3′
and

(SEQ ID NO: 10)

5′-GCTCCCATTGCTTTGGCCAC-3′;
Serum albumin (SAP):

(SEQ ID NO: 11)

5′-ATGCCTGCTGACTTGCCTTC-3′
and

(SEQ ID NO: 12)

5′-CTGAGGCTCTTCCACAAGAG-3′;
Phosphoglucomutase (PGM):

(SEQ ID NO: 13)

5′-AAGAAGATCCTCTGTGAAGAAC-3′
and

(SEQ ID NO: 14)

5′-GAATGCTGAAGATGTTGGCAG-3′;
Phenylalanine 4-hydroxylase (P4H):

(SEQ ID NO: 15)

5′-GGTGCCACTGTCCATGAGC-3′
and

(SEQ ID NO: 16)

5′-GGCGGTAGTTGTAGGCAATG-3′;
Apolipoprotein A1 (APOA1):

(SEQ ID NO: 17)

5′-GACAGCGGCAGAGACTATG-3′
and

(SEQ ID NO: 18)

5′-CCACCTTCTGGCGGTAGAG-3′;
Carbonic anhydrase III (CAIII):

(SEQ ID NO: 19)

5′-TCCTGACCACTGGCATGAAC-3′
and

(SEQ ID NO: 20)

5′-TGCTCAGAGCCATGATCATC-3′;
Mitochondrial aldehyde dehydrogenase (AdDHm):

(SEQ ID NO: 21)

5′-GAGCAGCAACCTCAAGAGAG-3′
and

(SEQ ID NO: 22)

5′-CGAGGATCTTCTTAAACTGAG-3′;
Ornithine aminotransferase (OAT):

(SEQ ID NO: 23)

5′-TGTGTCTGCAGTGCTGTGTG-3′
and

(SEQ ID NO: 24)

5′-AGACACACCTTCCAAGCATC-3′;
Ornithine carbamoyltransferase (OCT):

(SEQ ID NO: 25)

5′-GGAACAATATCCTGCACTCC-3′
and

(SEQ ID NO: 26)

5′-CTGTAATTAATACATTGCCTCC-3′;
Malate dehydrogenase:

(SEQ ID NO: 27)

5′-GGAGCAGCTGGTCAAATTGC-3′
and

(SEQ ID NO: 28)

5′-TCCATGCCTTCCCTTCTTGG-3′;
Glycine N-methyltransferase:

(SEQ ID NO: 29)

5′-GCTGGTGGAAGAGGGCTTC-3′
and

(SEQ ID NO: 30)

5′-CCTTTGCAGTCTGGCAAGTG-3′;
Apolipoprotein F:

(SEQ ID NO: 31)

5′-GTTGCTGGTCACATTCCTGG-3′
and

(SEQ ID NO: 32)

5′-TAGGCCTTCAACTCCTTCATG-3′;
Glutathione 5-transferase P2:

(SEQ ID NO: 33)

5′-AGTTCCAGGACGGAGACCTC-3′
and

(SEQ ID NO: 34)

5′-CAGGGTCTCAAAAGGCTTCAG-3′;
Methionine adenosyltransferase I:

(SEQ ID NO: 35)

5′-AGTCATCCCTGTGCGCATC-3′
and

(SEQ ID NO: 36)

5′-GGTCCACCTTGGTGTAGTC-3′;
Disulfide isomerase protein:

(SEQ ID NO: 37)

5′-TCTCCAAATACCAGCTCGAC-3′
and

(SEQ ID NO: 38)

5′-CAGGATCTTGCCCTTGAAGC-3′;
Adenosylhomocysteinase:

(SEQ ID NO: 39)

5′-GTCCAGCTGCAACATCTTCTC-3′
and

(SEQ ID NO: 40)

5′-CAGTCGTGGTCTCCTCAGAG-3′;
71-kDa thermal shock protein:

(SEQ ID NO: 41)

5′-GATGAAGGAAATTGCAGAAGC-3′
and

(SEQ ID NO: 42)

5′-CAATAGTGAGGATTGACACATC-3′;
Arginase 1:

(SEQ ID NO: 43)

5′-CAGGATTCTCCTGGGTGAC-3′
and

(SEQ ID NO: 44)

5′-GATGTAGAGACCTTCTCTG-3′;
Selenium-binding proteins:

(SEQ ID NO: 45)

5′-CTTCAGCAACTGGCTTGCATG-3′
and

(SEQ ID NO: 46)

5′-GCTGAGCTGGATCATCTGAG-3′;
Glutamine synthase:

(SEQ ID NO: 47)

5′-AGCCCAAGTGTGTGGAAGAG-3′
and

(SEQ ID NO: 48)

5′-TGCTGGTTGCTCACCATGTC-3′;
Glutathione S-transferase Mu1:

(SEQ ID NO: 49)

5′-CCTGGAATACACAGACTCAAG-3′
and

(SEQ ID NO: 50)

5′-TCTTCTCCTCTTCTGTCTCC-3′.

The specificity of the PCR products has been analysed by means of denaturation curves and electrophoresis. The transcript amount has been calculated and expressed as the relative difference with respect to the H3F3A Histone control gene (2^{ΔCt}, where ΔCt represents the difference in cycle number between the target gene and the control gene), as it has been previously described (K. J. Livak, T. D. Schmittgen. Analysis of Relative Gene Expression Data Using Real-Time Quantitative PCR and the 2^{-ΔCT} Method. Methods 2001; 25:402-408). A mixture of RNA from 5 control individuals was used as a positive control for the reaction, and milliQ water was used as a negative control.

### Biostatistical Analysis

In order to compare the mRNA levels between control individuals and patients with liver disease, the statistical test applied was the Mann-Whitney U test with a significance value of p<0.05.

### Results

### 1.1 Proteomic Profile of HCC in the Liver of the MAT1A−/− Mouse

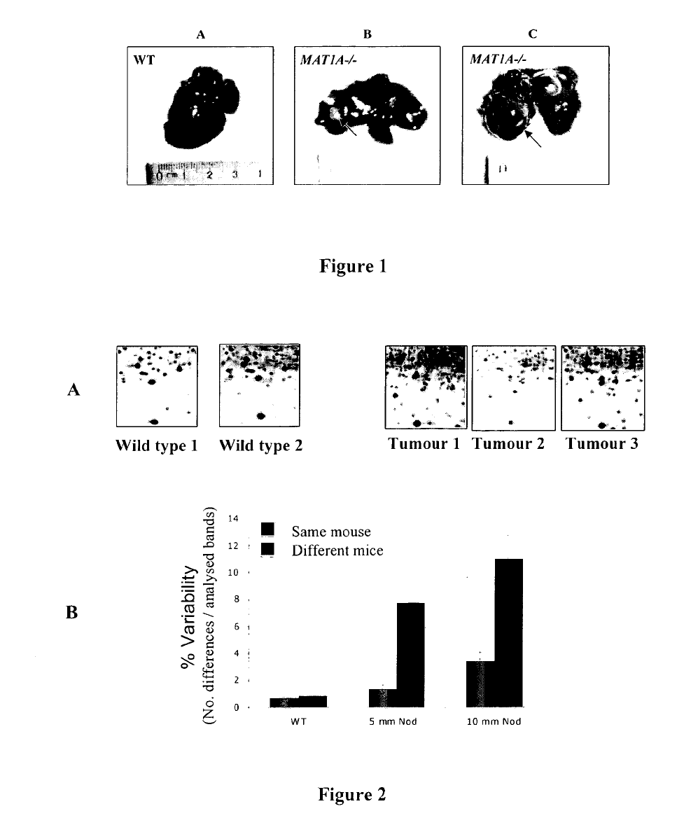

100% of the MAT1A−/− mice develop HCC at 18 months of age. The transformed hepatocytes are characterised in that they exhibit an oval nucleus and a prominent nucleolus. These hepatocytes form trabecular strands coated with endothelial cells. Macroscopic analysis of the tumour nodules present in different MAT1A−/− mice disclosed heterogeneity in the size thereof (FIG. 1).

The proteomic homogeneity that was observed in each of the analysed groups is one of the key parameters in differential proteomic studies. In this study, the measured variability (defined as the percentage of differential bands with respect to the total number of analysed bands) between livers of WT mice was 1% (p<0.01). However, upon comparing tumour nodules of the same MAT1A−/− liver, proteome variability reached 3% (p<0.01) in lesions greater than 10 mm and increased to 10% when nodules from different mice were compared, regardless of the size of the tumour (p<0.05). These data suggest that tumour growth as well as individual-specific factors may contribute to proteome instability following neoplastic transformation (FIG. 2).

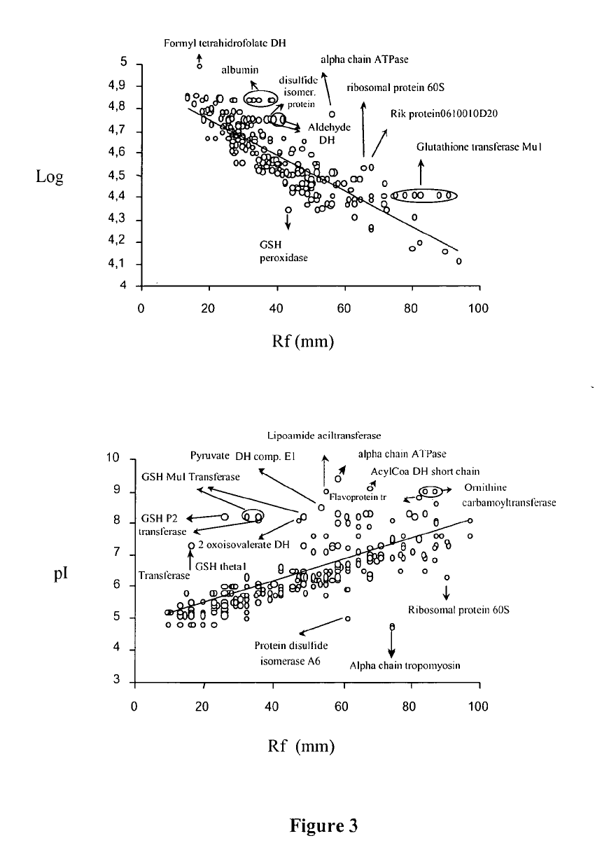

In order to define the changes in the liver proteome associated with hepatocarcinogenesis, liver samples from 18-month-old MAT1A−/− and WT mice were analysed by means of two-dimensional electrophoresis and mass spectrometry. All the analyses were performed in duplicate and the only differences that were considered as such were those proteins which exhibited a difference in expression of, at least, 3-fold in both replicas. 6 tumour nodules of different mice were compared to WT liver samples. Amongst all the alterations detected in the tumour nodules, 151 proteins were identified. In order to verify the identifications obtained, a representation was made of the theoretical Mw and pI calculated on the basis of the protein sequence, versus the experimental Rf of the corresponding bands calculated on the basis of the 2D gels. As it can be observed in FIG. 3, a good correlation between the theoretical and experimental parameters was obtained, with some deviations in both the experimental Mw and pI, probably due to post-translational modification or an alternative processing of the protein species. Some examples of anomalous electrophoretic behaviour observed in some tumours are: aldehyde dehydrogenase (class 2), albumin and glutathione transferase Mu1. In order to confirm the correct identification of these proteins, they were partially sequenced by means of ESI/MS/MS mass spectrometry.

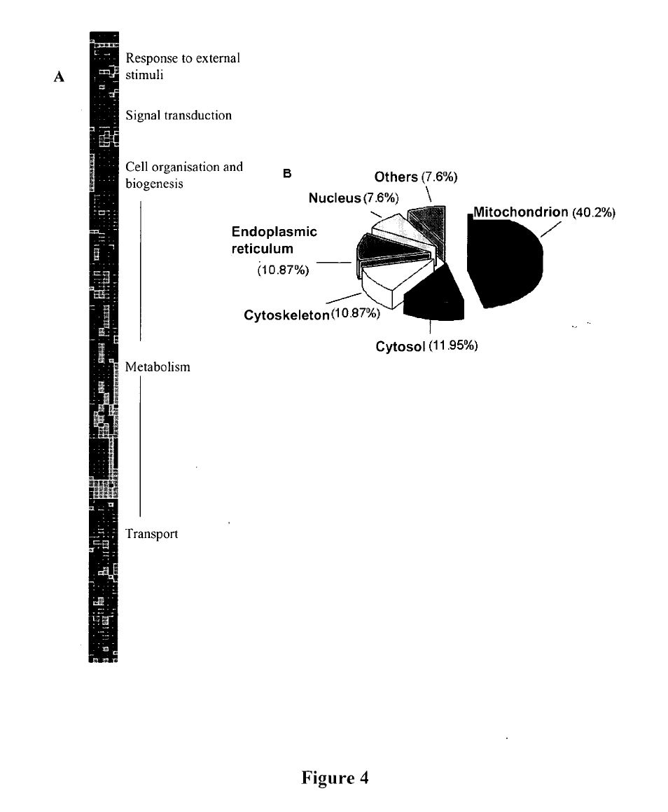

The identified proteins were classified on the basis of their subcellular location and the biological process wherein they are involved, in accordance with the criterion of Gene Ontology (Ashburner M, Ball C A, Blake J A et al. Gene Ontology: Tool for the Unification of Biology. The Gene Ontology Consortium. Nat. Genet. 2000; 25: 25-9), using the GARBAN biocomputer programme. The results obtained are shown in FIG. 4, where one can observe that the identified proteins are located in mitochondria (for the most part), the cytosol, the cytoskeleton, the endoplasmic reticulum, the nucleus and other subcellular locations. The biological processes wherein the identified proteins are involved comprise response to external stimuli, signal transduction, cell organisation and biogenesis, metabolism (for the most part) and transport.

Of the 151 proteins identified, 61.3% of the alterations correspond to proteins the levels thereof were reduced in the tumour, probably as a consequence of the progressive dedifferentiation of the hepatocytes during the transformation process. Despite the disparity observed between the different analyses, signalling, cell organisation, metabolism and transport were the commonly affected biological functions. The analyses indicated that the cytoskeleton and the endoplasmic reticulum were damaged, although the mitochondrion was the subcellular organelle accumulating the largest number of alterations. Study of the metabolic alterations undergone in the MAT1A−/− liver indicated that, as was the case in non-alcoholic steatohepatitis (NASH), most of them pertained to the metabolism of carbohydrates, lipids and amino acids. Furthermore, an increase was observed in the expression of enzymes related to the metabolism of purines and pyrimidines. This observation might reflect the need for synthesis of genetic material by proliferating cells.

In order to identify proteins associated with human HCC, it was decided to focus the study solely on the 27 proteins the expression thereof was altered in, at least, 50% of the analysed MAT1A−/− tumours (Table 1).

TABLE 1
HCC-associated alterations in at least 50% of the analysed tumour nodules

| Protein | Code | Access no. | Function | Cases |
|---|---|---|---|---|
| Methionine | MAT | Q91X83 | Adomet synthesis | 6\6 |
| adenosyltransferase | | | | |
| Carbonic anhydrase III | CAIII | P16015 | Reversible hydration of | 6\6 |
| | | | carbon dioxide | |
| 1300018L09 Rik protein | Q9DBB8 | Q9DBB8 | Polycyclic hydrocarbon | 5\6 |
| | | | catabolism | |
| Glutamine synthetase | GS | P15105 | Nitrogen homeostasis | 5\6 |
| Selenium-binding protein 2 | SBP2 | Q63836 | Binding to Selenium | 5\6 |
| | | | and acetaminophen | |
| Adenosylhomocysteinase | SAHH | P50247 | Regulates S- | 5\6 |
| | | | adenosylhomocysteine | |
| | | | levels | |
| Antioxidant protein 2 | AOP2 | O08709 | Involved in redox | 5\6 |
| | | | regulation of the cell | |
| Regucalcin | SMP30 | Q64374 | Calcium homeostasis | 5\6 |
| Apolipoprotein A1 | APOA1 | Q00623 | Reverse transport of | 4\6 |
| | | | cholesterol to the liver | |
| Apolipoprotein E | APOE | P08226 | Binding and | 4\6 |
| | | | internalisation of | |
| | | | lipoproteins | |
| Albumin | SAP | P07724 | Regulates blood | 4\6 |
| | | | osmotic pressure | |
| Sorbitol dehydrogenase | SDH | Q64442 | Involved in the polyol | 3\6 |
| | | | pathway | |
| Phenylalanine 4- | P4H | P16331 | Phenylalanine | 3\6 |
| hydroxylase | | | catabolism | |
| Malate dehydrogenase | MDH | P14152 | Involved in the Krebs | 3\6 |
| | | | cycle | |
| Arginase 1 | ARG | Q61176 | Arginine degradation | 3\6 |
| | | | (Urea Cycle) | |
| Phosphoglucomutase | PGM | Q9D0F9 | Glucose | 3\6 |
| | | | degradation/synthesis | |
| Ornithine aminotransferase | OAT | P29758 | Controls L-ornithine | 3\6 |
| | | | levels in tissues | |
| Ornithine | OCT | P00481 | Participates in Arginine | 3\6 |
| carbamoyltransferase | | | biosynthesis | |
| Sterol-carrying protein 2 | NSLT | P32020 | Transports Acyl-CoA | 3\6 |
| | | | groups | |
| Glycine N- | GNMT | Q91WN7 | Glycine methylation | 3\6 |
| methyltransferase | | | | |
| Alcohol dehydrogenase | ADH | Q9JII6 | Ethanol metabolism | 3\6 |
| 2810435D12 Rik protein | Q9CYW4 | Q9CYW4 | Hydrolase activity | 3\6 |
| Mit. aldehyde | AdDHm | P44738 | Involved in ethanol | 3\6 |
| dehydrogenase | | | utilisation | |
| Glutathione transferase Mu1 | GST Mu1 | P10649 | Detoxification of | 3\6 |
| | | | hydrophobic | |
| | | | electrophiles | |
| Glutathione transferase P2 | GST P2 | P46425 | Detoxification of | 3\6 |
| | | | hydrophobic | |
| | | | electrophiles | |
| 71 kDa thermal shock | HSC71 | P63017 | Stress protein | 3\6 |
| protein | | | | |
| Disulfide isomerase protein | PDI | P09103 | Chaperonin, isomerase | 3\6 |
| | | | with redox activity | |

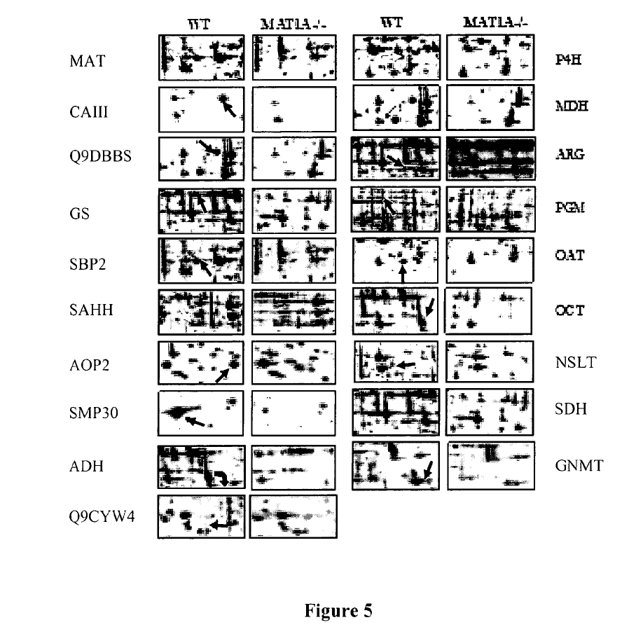

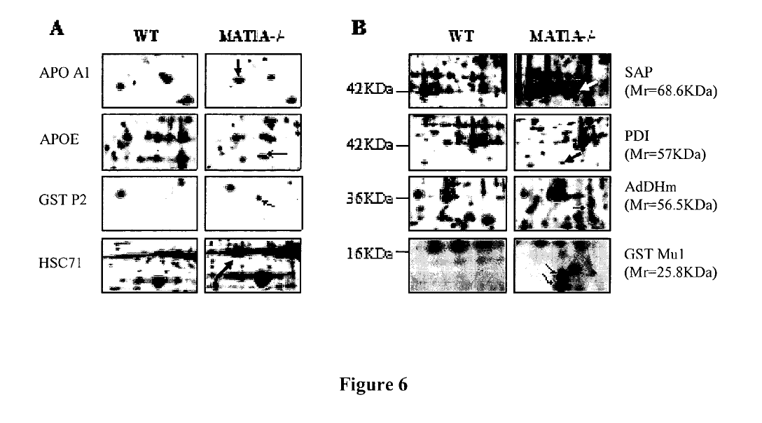

Of the 27 proteins, 19 reduced their expression in the MAT1A−/− liver (methionine adenosyltransferase, carbonic anhydrase III, 1300018L09 Rik protein, glutamine synthetase, selenium-binding protein 2, adenosylhomocysteinase, antioxidant protein 2, regucalcin, sorbitol dehydrogenase, phenylalanine 4-hydroxylase, malate dehydrogenase, arginase 1, phosphoglucomutase, ornithine aminotransferase, ornithine carbamoyltransferase, sterol-carrying protein 2, glycine N-methyltransferase, alcohol dehydrogenase and 2810435D12 Rik protein) (FIG. 5). The remaining 8 proteins (glutathione transferase P2, 71 kDa heat shock protein, apolipoprotein A1 and E, albumin, class 2 aldehyde dehydrogenase, glutathione transferase Mu1 and disulfide isomerase protein) were increased in the analysed tumours. Four of them were analysed on the basis of newly appeared bands, which exhibited anomalous electrophoretic behaviour due to changes observed in both the pI and Mw. One cannot conclude that these proteins are overexpressed, since these anomalous isoforms are possibly produced as a consequence of post-translational modifications (FIG. 6).

### 1.2 Analysis of Gene Expression in Human HCC

In order to investigate whether the previously observed alterations in the MAT1A−/− mouse can be considered to be potential HCC biomarkers in humans, the levels of 23 of the 27 proteins were analysed in ten HCC of various etiologies, by means of the quantitative PCR technique. The reduction in the expression of methionine adenosyltransferase and glycine N-methyltransferase had been previously demonstrated at the inventors' laboratory (Ávila M A, Berasain C, Torres L, Martín-Duce A, Corrales F J, Yang H, Prieto J, Lu S C, Caballería J, Rodes J, Mato J M. Reduced ARNm Abundance of the Main Enzymes Involved in Methionine Metabolism in Human Liver Cirrhosis and Hepatocellular Carcinoma. J Hepatol 2000; 33: 907-914). The 1300018L09 and 2810435D12 Rik proteins were not included in this study since their assignment in databases is due to sequence homology with other proteins and, therefore, their function is yet to be experimentally demonstrated. In order to verify the designed primer oligonucleotides, 23 RT-PCR reactions were performed on a mixture of human tumour cell lines and it was verified that the expression of all of them decreased with respect to control livers. The transcript amount was expressed as the relative difference with respect to the H3F3A Histone control gene (2^{ΔCt}, where ΔCt represents the difference in cycle number between the target gene and the control gene). The Mann-Whitney U statistical test, with a significance value of p<0.05, was applied in all the analyses conducted. The expression analyses performed by means of RT-PCR showed that 13 of the 23 proteins exhibited significant alterations with respect to the control group. These proteins were: alcohol dehydrogenase, antioxidant protein 2, regucalcin, sterol-carrying protein 2, sorbitol dehydrogenase, albumin, phosphoglucomutase, phenylalanine 4-hydroxylase, apolipoprotein A1, carbonic anhydrase III, class 2 aldehyde dehydrogenase, ornithine aminotransferase and ornithine carbamoyltransferase. In order to investigate whether the observed alterations were exclusive of HCC or were present in a pre-tumour stage such as cirrhosis, the expression levels of the 13 altered proteins (proteins having an altered expression pattern) were analysed by means of RT-PCR in samples from cirrhotic patients of both alcoholic etiology (n=5) and viral etiology (n=5) who had not developed a tumour. The expression analyses reflected that the expression of 7 of the 13 proteins (alcohol dehydrogenase, antioxidant protein 2, regucalcin, sterol-carrying protein 2, sorbitol dehydrogenase, albumin and phosphoglucomutase) was significantly altered in patients with cirrhosis with respect to the control group. These alterations were also detected in 5 samples of peritumoural tissue from individuals suffering from viral cirrhosis and HCC. Regardless of whether or not the analysed tissue was associated with a tumour, no significant differences were observed; therefore, it was decided to group the patients with viral cirrhosis (n=10) in subsequent studies. Subsequently, the expression of the 13 proteins was analysed in peritumoural tissue samples from individuals with HCC and without cirrhosis. To this end, measurements were taken, by means of RT-PCR, of the expression levels in samples from patients with hepatitis (n=3) and in samples from patients who had developed HCC on a healthy liver (n=7). Alcohol dehydrogenase was the only protein wherein significant expression differences were observed with respect to the control group (n=6).

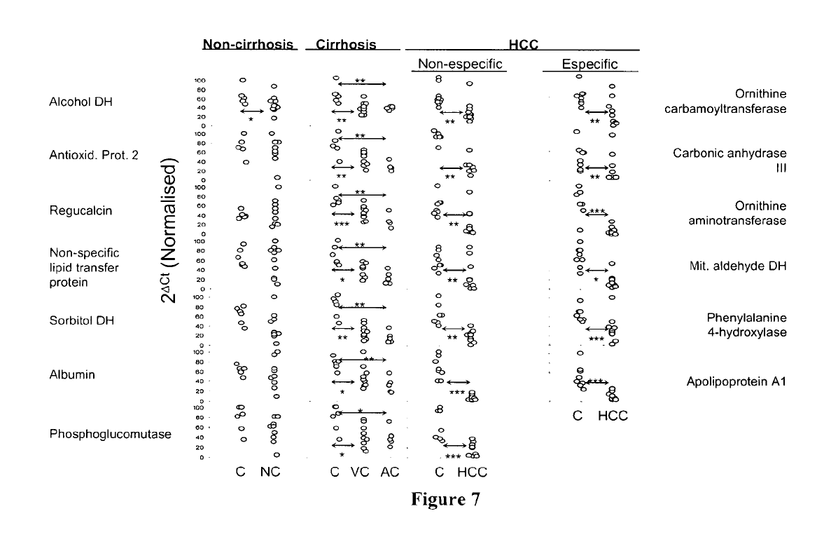

In sum, proteomic analysis of HCC in the MAT1A−/− mouse made it possible to identify 27 altered proteins (proteins having an altered expression pattern) in, at least, 50% of the studied cases. Amongst them, 13 were detected in human hepatocarcinomas by means of quantitative PCR. Finally, the identification of 7 of the 13 alterations in peritumoural cirrhosis or cirrhosis not associated with HCC suggests the utility of this panel of potential biomarkers (FIG. 7), not only in the diagnosis, but also in the early detection of pre-tumour stages.

### Discussion

The comparison of WT liver proteomes and tumour nodules of the MAT1A−/− mouse has made it possible to identify 151 differential proteins which provide a proteomic imprint of HCC, setting a molecular basis for some of the alterations which characterise the tumour biology. The variability in the group of identified differential proteins is a reflection of the proteomic heterogeneity of the tumour nodules. Furthermore, it was observed that 61.3% of the alterations pertain to proteins the levels thereof are reduced in the tumour, probably as a consequence of the progressive dedifferentiation of the hepatocytes during the transformation process. Functional analysis of the observed alterations indicates that, in the transformed hepatocytes, there is a decrease in metabolic activity, as well as an increase in proteins associated with stress response, which could be considered to be an adaptation to the pressure imposed by the deficiency of AdoMet. The differential proteins are preferentially located in the mitochondrion, the cytoplasm, the endoplasmic reticulum and the cytoskeleton. Despite the low coincidence level observed upon comparing the differential proteins or genes, the metabolic and respiratory deficiency, as well as the stress response, constitute a functional profile of HCC common to different studies which use a genomic or proteomic approach (Xu X R, Huang J, Xu Z G, Qian B Z et al. Insight into Hepatocellular Carcinogenesis at Transcriptome Level by Comparing Gene Expression Profiles of Hepatocellular Carcinoma with Those of Corresponding Noncancerous Liver. Proc Natl Acad Sci USA 2001; 98: 15089-94; Cynthia R M Yliang, Chon Kar Leow et al. Proteome Analysis of Human Hepatocellular Carcinoma Tissues by Two-Dimensional Difference Gel Electrophoresis and Mass Spectrometry. Proteomics 2005; 5: 2258-2271) and, therefore, one can rule out that they are specifically produced in the experimental model used. It is yet to be resolved whether these changes simply represent the functional alteration of the liver once the lesion is established or whether they actually participate in the pathogenesis of human HCC. However, the alterations of the liver metabolism and the decrease in mitochondrial activity, which has been proposed as one of the factors involved in the pathogenesis of several types of cancer, are produced in the liver of the MAT1A−/− mouse from birth, long before any manifestation of the disease is detected at the histological level, which suggests their participation in the development of HCC.

Of the 151 altered proteins (proteins having an altered expression pattern) in the liver of the MAT1A−/− mouse, 27 were detected as differences in, at least, 50% of the analysed tumours and, for this reason, were considered to be potential HCC markers. It is worth stressing that this group of alterations selected for their high incidence reproduce the previously described functional phenotype of the tumours: metabolic enzymes the alteration thereof represents a deficient metabolism of carbohydrates (phosphoglucomutase), amino acids (arginase 1, ornithine carbamoyltransferase), methyl group metabolism (methionine adenosyltransferase, adenosylhomocysteinase, glycine N-methyltransferase), lipid metabolism and transport (sterol-carrying protein 2, apolipoproteins A1 and E), and an increase in proteins involved in detoxification and stress response (71-kDA heat shock protein, disulfide isomerase protein, isoforms of glutathione transferases Mu1 and P2). Furthermore, the selenium-binding protein has been related to fibrosis and ageing, for which reason its reduction might favour the development of the tumour. The alteration of sorbitol dehydrogenase (SDH) activity has been used as an indicative parameter of liver damage. The SDH reduction in the tumour nodules of the MAT1A−/− mouse is correlated with the loss of the capacity to metabolise sorbitol in the transformed cells. Regucalcin (also called senescence-marking protein) is a calcium-binding protein that plays a decisive role in the maintenance of homeostasis and cell function in the liver. Finally, glutamine synthetase and ornithine aminotransferase are enzymes which are involved in glutamine metabolism, and the overexpression thereof, partly through the Wnt/β-catenine pathway, is associated with hepatic carcinogenesis. In rodents, it has been demonstrated that a positive glutamine synthetase phenotype favours tumour growth, although this alteration is not present in all the lesions. The reduction in glutamine synthetase levels in the tumours of the MAT1A−/− mouse might be a characteristic feature of this experimental model of AdoMet deficiency in the liver. However, the results obtained by the inventors do not allow to rule out potential post-translational modifications which may give rise to a new species that is not detectable under the analysis conditions used.

There are studies, both genomic and proteomic, wherein peritumoural tissue is used as a reference in order to detect changes in expression associated with HCC (Kim J W, Ye Q, Forgues M, Chen Y et al. Cancer-Associated Molecular Signature in the Tissue Samples of Patients with Cirrhosis. Hepatology 2004; 39: 518-27; Xu X R, Huang J, Xu Z G, Qian B Z et al. Insight into Hepatocellular Carcinogenesis at Transcriptome Level by Comparing Gene Expression Profiles of Hepatocellular Carcinoma with Those of Corresponding Noncancerous Liver. Proc Natl Acad Sci USA 2001; 98: 15089-94; Lim S O, Park S J, Kim W, Park S G, Kim H J et al. Proteome Analysis of Hepatocellular Carcinoma. Biochem Biophys Res Commun 2002; 291: 1031-37; Kim J, Kim S H, Lee S U, Ha G H, Kang D G et al. Proteome Analysis of Human Liver Tumour Tissue by Two Dimensional Gel Electrophoresis and Matrix-Assisted Laser Desorption/Ionisation Mass Spectrometry for Identification of Disease-Related Proteins. Electrophoresis 2002; 23: 4142-4156; Kim W, Lim S O, Ryu Y H, Byeon J Y et al. Comparison of Proteome between Hepatitis B Virus and Hepatitis C Virus Associated Hepatocellular Carcinoma. Clin Cancer Res 2003; 9: 5493-5500; Li C, Hong Y, Tan Y X, Zhou H, Ai J H et al. Accurate Quality and Quantitative Proteomic Analysis of Clinical Hepatocellular Carcinoma Using Laser Capture Microdissection Coupled with Isotope-Coded Affinity Tag and Two Dimensional Liquid Chromatography Mass Spectrometry. Mol Cell Proteomics 2004; 3: 399-409; Li C, Tan Y X, Zhou H, Ding S J, Li S J et al. Proteomic Analysis of Hepatitis B Virus-Associated Hepatocellular Carcinoma Identification of Potential Tumour Markers. Proteomics 2005; 5: 1125-1135). This strategy would not allow for the detection of alterations common to the non-tumour part and to the tumour, preventing the identification of those proteins the expression thereof initially changes in hepatitis or in cirrhosis, stages prior to the development of HCC.

The use of the MAT1A−/− mouse as a screening system prior to the analysis of human samples has proven to be effective in the identification of HCC markers. The proteomic complexity is reduced to 27 potential markers deduced from the analyses conducted with the MAT1A−/− mouse, of which 13 have been validated in human HCC by means of quantitative PCR studies. The reduction in ornithine carbamoyltransferase, ornithine aminotransferase, mitochondrial class 2 aldehyde dehydrogenase, carbonic anhydrase III, apolipoprotein A1 and phenylalanine 4-hydroxylase was only observed in tumours and, therefore, they could be considered to be markers of neoplastic transformation. However, the alterations in alcohol dehydrogenase, antioxidant protein 2, regucalcin, sterol-carrying protein 2, sorbitol dehydrogenase, albumin and phosphoglucomutase, as well as in methionine adenosyltransferase and glycine N-methyltransferase were detected in the cirrhotic liver, regardless of whether or not the analysed tissue was associated with a tumour. Cirrhosis is considered to be a high-risk situation for the development of HCC and, therefore, these alterations, which remain in the tumour, could represent early markers that would allow for the definition of a preneoplastic stage. The analysis of these markers in healthy tissue samples or Hepatitis C virus- or Hepatitis B virus-infected tissue from livers with HCC was negative, with the sole exception of alcohol dehydrogenase, which has been previously associated with liver lesion processes of very different etiology. In sum, a repertoire of potential HCC markers is described, some early and other tumour-specific, which might be of great utility in monitoring the population at risk and in the early detection of this disease.

1. An in vitro method to diagnose hepatocellular carcinoma (HCC) in a subject, or to evaluate a subject's predisposition to develop HCC, or to evaluate the progression of HCC in a subject, or to determine the stage or severity of HCC in a subject, comprising:
a) quantifying the expression level of a gene in a liver tissue sample from said subject, wherein said gene is chosen from the gene that encodes alcohol dehydrogenase (ADH) [ADH gene], the gene that encodes antioxidant protein 2 (AOP2) [AOP2 gene], the gene that encodes regucalcin (SMP30) [SMP30 gene], the gene that encodes sterol-carrying protein 2 (NSLT) [NSLT gene], the gene that encodes sorbitol dehydrogenase (SDH) [SDH gene], the gene that encodes albumin (SAP) [SAP gene], the gene that encodes phosphoglucomutase (PGM) [PGM gene], the gene that encodes phenylalanine 4-hydroxylase (P4H) [P4H gene], the gene that encodes apolipoprotein A1 (APOA1) [APOA1 gene], the gene that encodes carbonic anhydrase III (CAIII) [CAIII gene], the gene that encodes class 2 aldehyde dehydrogenase (AdDHm) [gene 30AdDHm], the gene that encodes ornithine aminotransferase (OAT) [OAT gene], the gene that encodes ornithine carbamoyltransferase (OCT) [OCT gene], and combinations thereof; and
b) comparing the expression level to that of a control sample;
wherein a reduction in the expression level of the sample with respect to the expression level in the control sample is indicative of HCC;
or, alternatively,
a) quantifying the level of a protein in a liver tissue sample from said subject, wherein said protein is chosen from alcohol dehydrogenase (ADH), antioxidant protein 2 (AOP2), regucalcin (SMP30), sterol-carrying protein 2 (NSLT), sorbitol dehydrogenase (SDH), albumin (SAP), phosphoglucomutase (PGM), phenylalanine 4-hydroxylase (P4H), apolipoprotein A1 (APOA1), carbonic anhydrase III (CAIII), class 2 aldehyde dehydrogenase (AdDHm), ornithine aminotransferase (OAT), ornithine carbamoyltransferase (OCT), and combinations thereof; and
b) comparing the level of the protein in the sample to that of a control sample;
wherein a reduction in the level of protein in the sample with respect to the level in the control sample is indicative of HCC.

2-37. (canceled)
